(19) United States
(12) Patent Application Publication
(10) Pub. No.: US 2010/0010022 A1
(43) Pub. Date: Jan. 14, 2010

(54) CANCER TREATMENT METHOD

(75) Inventors: Rakesh KUMAR, Durham, NC (US); Robert John MULLIN, Durham, NC (US); Tona M. GILMER, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SmithKline Beecham Corporation, Philadelphia, PA (US)

(21) Appl. No.: 12/536,302

(22) Filed: Aug. 5, 2009

Related U.S. Application Data
(63) Continuation of application No. 10/599,967, filed on Oct. 16, 2006.
(60) Provisional application No. 60/605,288, filed on Aug. 27, 2004.
(60) Provisional application No. 60/563,285, filed on Apr. 16, 2004.

(51) Int. Cl. A61K 31/517; A61K 31/506; A61P 35/00

(52) U.S. Cl. 514/266.24; 514/275; 514/266.2

(57) ABSTRACT

A method of treating cancer is described including administration of a pyrimidine derivative and a quinazoline derivative as well as a pharmaceutical composition including the same.

### CROSS-REFERENCE TO RELATED APPLICATIONS

This application is a continuation of U.S. patent application Ser. No. 10/599,967 filed Oct. 16, 2006; which was filed pursuant to 35 U.S.C. §371 as a U.S. National Phase Application of International Application No. PCT/US2005/012337 filed Apr. 12, 2005; which claims priority from U.S. Provisional Application No. 60/563,285 filed Apr. 16, 2004 and U.S. Provisional Application No. 60/605,288 filed Aug. 27, 2004.

### FIELD OF THE INVENTION

The present invention relates to pharmaceutical combinations and methods of treating cancer utilizing the same. Specifically, the invention relates to a combination of an inhibitor of VEGFR, and an inhibitor of Erb-B2 and/or Erb-B1, as well as use of the combination in the treatment of cancer.

### BACKGROUND OF THE INVENTION

Effective treatment of hyperproliferative disorders, including cancer, is a continuing goal in the oncology field. Protein tyrosine kinases catalyse the phosphorylation of cell growth and differentiation of specific tyrosyl residues in various proteins involved in the regulation of cell growth and differentiation. (A. F. Wilks, Progress in Growth Factor Research, 1990, 2, 97-111; S. A. Courtneidge, Dev. Supp. I, 1993, 57-64; J. A. Cooper, Semin. Cell Biol., 1994, 5(6), 377-387; R. F. Paulson, Semin. Immunol., 1995, 7(4), 267-277; A. C. Chan, Curr. Opin. Immunol., 1996, 8(3), 394-401). Inappropriate or uncontrolled activation of many of such kinases, i.e., aberrant protein tyrosine kinase activity, for example by over-expression or mutation, has been shown to result in uncontrolled cell growth.

In cancer the growth of solid tumors has been shown to be dependent on angiogenesis. The progression of leukemias as well as the accumulation of fluid associated with malignant ascites and pleural effusions also involve pro-angiogenic factors. (See Folkmann, J., J. Nat'l. Cancer Inst., 1990, 82, 4-6.) Consequently, the targeting of pro-angiogenic pathways is a strategy being widely pursued in order to provide new therapeutics in these areas of great, unmet medical need.

Central to the process of angiogenesis are vascular endothelial growth factor (VEGF) and its receptors, termed vascular endothelial growth factor receptor(s) (VEGFRs). Three PTK receptors for VEGF have been identified: VEGFR-1 (Flt-1); VEGFR-2 (Flk-1 and KDR) and VEGFR-3 (Flt-4). These receptors are involved in angiogenesis and participate in signal transduction. (Mustonen, T. et al J. Cell Biol. 1995:129:895-898; Ferrara and Davis-Smyth, Endocrine Reviews, 18(1):4-25, 1997; McMahon, G., The Oncologist, Vol. 5, No 90001, 3-10, April 2000).

Of particular interest is VEGFR-2, which is a transmembrane receptor PTK expressed primarily in endothelial cells. Activation of VEGFR-2 by VEGF is a critical step in the signal transduction pathway that initiates tumor angiogenesis.

Consequently, antagonism of the VEGFR-2 kinase domain would block phosphorylation of tyrosine residues and serve to disrupt initiation of angiogenesis, thereby providing a potent treatment for cancer or other disorders associated with inappropriate angiogenesis.

The erbB family of protein tyrosine kinases is another group of kinases that has been implicated in human malignancies. Elevated Erb-B1 (EGFR) receptor activity has, for example, been implicated in non-small cell lung, bladder, renal cell, and head and neck cancers. Increased c-Erb-B2 activity is associated with breast, ovarian, gastric and pancreatic cancers. Consequently, inhibition of such protein tyrosine kinases should provide a treatment for disorders characterized by aberrant Erb family protein kinase activity.

International Patent Application PCT/US01/49367 filed Dec. 19, 2001, and published as WO 02/059110 on Aug. 1, 2002, discusses PTKs including VEGFR family PTKs. This published application discloses bicyclic heteroaromatic compounds, including 5-({4-[(2,3-dimethyl-2H-indazol-6-yl)(methyl)amino]pyrimidin-2-yl}amino)-2-methylbenzenesulfonamide; as well as hydrochloride salts thereof. These compounds show inhibition activity against VEGFR-2. International Patent Application PCT/EP99/00048 filed Jan. 8, 1999, and published as WO 99/35146 on Jul. 15, 1999, discusses PTKs including ErbB family PTKs. This published application discloses bicyclic heteroaromatic compounds, including N-{3-Chloro-4-[(3-fluorobenzyl)oxy]phenyl}-6-[5-({[2-(methanesulphonyl)ethyl]amino}methyl)-2-furyl]-4-quinazolinamine; (4-(3-Fluoro-benzyloxy)-3-chlorophenyl)-(6-(2-((2-methanesulphonyl-ethylamino)-methyl)-thiazol-4-yl)quinazolin-4-yl)-amine; (4-(3-Fluoro-benzyloxy)-3-bromophenyl)-(6-(5-((2-methanesulphonyl-ethylamino)-methyl)-furan-2-yl)quinazolin-4-yl)-amine as well as hydrochloride salts thereof. These compounds show inhibition activity against erbB family PTKs.

Combination therapy is rapidly becoming the norm in cancer treatment, rather than the exception. Oncologists are continually looking for anti-neoplastic compounds which when utilized in combination provides a more effective and/or enhanced treatment to the individual suffering the effects of cancer. Typically, successful combination therapy provides improved and even synergistic effect over monotherapy.

Combination of VEGF and ErbB inhibitors have been explored in several pre-clinical tumor models (Ciardiello F. et al. Clin Cancer Res 2000; 6(9):3739-3747; Baker C H et al. Cancer Res 2002; 62(7):1996-2003; Shaheen R M et al. Br J Cancer 2001; 85(4):584-589; Jung Y D et al. Eur J Cancer 2002; 38(8):1133-1140). In mice bearing human colon carcinoma xenografts, combined treatment with anti-EGFR mAb (c225) and VEGF antisense significantly improved survival compared to either agent alone (Ciardiello F. et al. Clin Cancer Res 2000; 6(9):3739-3747). Similarly, combination of antibodies against Erb-B1 and VEGF receptor resulted in decreased angiogenesis and ascites formation compared to either antibody alone in a mouse model of peritoneal carcinomatosis (Baker C H et al. Cancer Res 2002; 62(7):1996-2003).

The present inventors have now identified combinations of chemotherapeutic agents that provide increased activity over monotherapy. In particular, multiple drug combinations that include inhibitors of the VEGFR family of kinases in combination with inhibitors of the ErbB family of kinases are described.

### SUMMARY OF THE INVENTION

Briefly, one aspect of the present invention provides a method of treating cancer in a mammal, comprising:

administering to said mammal
(a) a compound of formula I

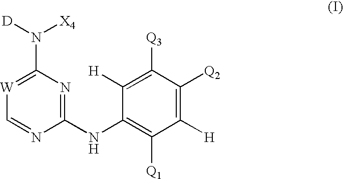

or a salt, solvate, or physiologically functional derivative thereof;
wherein

D is

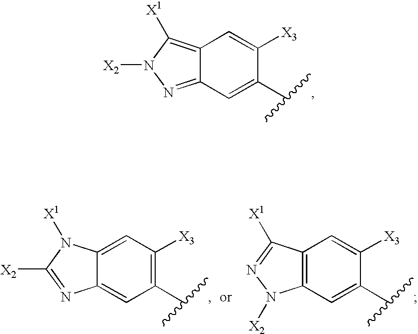

X₁ is hydrogen, C₁-C₄ alkyl, C₁-C₄ haloalkyl, or C₁-C₄ hydroxyalkyl;
X₂ is hydrogen, C₁-C₄ alkyl, C₁-C₄ haloalkyl, C(O)R¹, or aralkyl;
X₃ is hydrogen or halogen;
X₄ is hydrogen, C₁-C₄ alkyl, C₁-C₄ haloalkyl, heteroaralkyl, cyanoalkyl, —(CH₂)ₚC═CH(CH₂)ₜH, —(CH₂)ₚC≡C(CH₂)ₜH, or C₃-C₇ cycloalkyl;
p is 1, 2, or 3;
t is 0 or 1;
W is N or C—R, wherein R is hydrogen, halogen, or cyano;
Q₁ is hydrogen, halogen, C₁-C₂ haloalkyl, C₁-C₂ alkyl, C₁-C₂ alkoxy, or C₁-C₂ haloalkoxy;

### Q₂ is A¹ or A²;

Q₃ is A¹ when Q₂ is A² and Q₃ is A² when Q₂ is A¹;
wherein

A¹ is hydrogen, halogen, C₁-C₃ alkyl, C₁-C₃ haloalkyl, —OR¹, and

A² is the group defined by -(Z)ₘ-(Z¹)-(Z²), wherein
Z is CH₂ and m is 0, 1, 2, or 3, or
Z is NR² and m is 0 or 1, or
Z is oxygen and m is 0 or 1, or
Z is CH₂NR² and m is 0 or 1;
Z¹ is S(O)₂, S(O), or C(O); and
Z² is C₁-C₄ alkyl, NR³R⁴, aryl, arylamino, aralkyl, aralkoxy, or heteroaryl;
   R¹ is C₁-C₄ alkyl;
   R², R³, and R⁴ are each independently selected from hydrogen, C₁-C₄ alkyl, C₃-C₇ cycloalkyl, —S(O)₂R⁵, and —C(O)R⁵;
   R⁵ is C₁-C₄ alkyl, or C₃-C₇ cycloalkyl; and
   when Z is oxygen then Z¹ is S(O)₂ and when D is

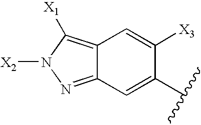

then X₂ is C₁-C₄ alkyl, C₁-C₄ haloalkyl, C(O)R¹, or aralkyl; and
(b) a compound of formula II

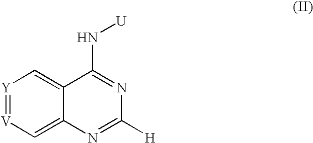

or a salt, solvate, or physiologically functional derivative thereof;
wherein

### Y is CR⁶ and V is N;

### or Y is CR⁶ and V is CR⁷;

R⁶ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R⁷ is selected from the group consisting of hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R⁸ group and optionally substituted by at least one independently selected R⁹ group;
R⁸ is selected from the group consisting of benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R⁸ represents trihalomethylbenzyl or trihalomethylbenzyloxy;
or R⁸ represents a group of formula

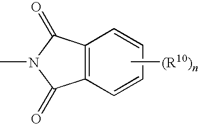

wherein each R¹⁰ is independently selected from halogen, C₁₋₄ alkyl and C₁₋₄ alkoxy; and n is 0 to 3; and
each R⁹ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro or trifluoromethyl.

In another aspect, the present invention provides a pharmaceutical composition comprising a compound of formula I or salt, solvate, or physiologically functional derivative thereof, and a compound of formula II or salt, solvate, or physiologically functional derivative thereof, optionally in association with a pharmaceutically acceptable diluent or carrier.

In a further aspect, the present invention provides a pharmaceutical combination comprising a compound of formula I or salt, solvate or physiologically functional derivative thereof and a compound of formula II or salt, solvate or physiologically functional derivative thereof for use in therapy.

In a further aspect, the present invention provides a pharmaceutical combination comprising a compound of formula I or salt, solvate or physiologically functional derivative thereof and a compound of formula II or salt, solvate or physiologically functional derivative thereof for the manufacture of a medicament for the treatment of cancer.

### BRIEF DESCRIPTION OF THE DRAWINGS

FIG. 1 depicts anti-tumor activity in a subcutaneous human xenograft mouse model dosed with a compound of Example 1 and a compound of Example 3 individually and in combination versus BT474 (human breast tumor line). The figure is the graphical representation of the data contained in Table 3.

FIG. 2 depicts anti-tumor activity in a subcutaneous human xenograft mouse model dosed with a compound of Example 1 and a compound of Example 3 individually and in combination versus NCI-H322 (non-small cell lung carcinoma). The figure is the graphical representation of the data contained in Table 4.

### DETAILED DESCRIPTION OF THE INVENTION

Terms are used within their accepted meanings. The following definitions are meant to clarify, but not limit the terms defined.

As used herein, “a compound of formula (X)” means a compound of formula X, or a salt, solvate, or physiological functional derivative thereof, wherein X is I, II or any number of the like. For example, a compound of formula I is a compound of formula I or a salt, solvate, or physiologically functional derivative thereof.

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “lower” refers to a group having between one and six carbons.

As used herein, the term “alkyl” refers to a straight or branched chain hydrocarbon having from one to twelve carbon atoms, optionally substituted with substituents selected from the group consisting of lower alkyl, lower alkoxy, lower alkylsulfanyl, lower alkylsulfenyl, lower alkylsulfonyl, oxo, mercapto, amino optionally substituted by alkyl, carboxy, carbamoyl optionally substituted by alkyl, aminosulfonyl optionally substituted by alkyl, nitro, or lower perfluoroalkyl, multiple degrees of substitution being allowed. Examples of “alkyl” as used herein include, but are not limited to, n-butyl, n-pentyl, isobutyl, and isopropyl, and the like.

As used herein, the term “alkylene” refers to a straight or branched chain divalent hydrocarbon radical having from one to ten carbon atoms, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and lower perfluoroalkyl, multiple degrees of substitution being allowed. Examples of “alkylene” as used herein include, but are not limited to, methylene, ethylene, n-propylene, n-butylene, and the like.

As used herein, the term “C_{x-y}” where x and y represent an integer value refer to the number of carbon atoms in a particular chemical term to which it is attached. For instance, the term “C₁₋₄ alkyl” refers to an alkyl group, as defined herein, containing at least 1, and at most 4 carbon atoms. The term “C₁₋₄ alkylene” refers to an alkylene group, as defined above, which contains at least 1, and at most 4 carbon atoms.

As used herein, the term “C₁₋₄ haloalkyl” refers to a straight or branched chain hydrocarbon containing at least 1, and at most 4, carbon atoms substituted with at least one halogen. Examples of branched or straight chained “C₁₋₄ haloalkyl” groups useful in the present invention include, but are not limited to, methyl, ethyl, propyl, isopropyl, isobutyl and n-butyl substituted independently with one or more halogens, e.g., fluoro, chloro, bromo and iodo.

As used herein, the term “C₁₋₄ hydroxyalkyl” refers to a straight or branched chain hydrocarbon containing at least 1, and at most 4, carbon atoms substituted with at least one hydroxy. Examples of branched or straight chained “C₁₋₄ hydroxyalkyl” groups useful in the present invention include, but are not limited to, methyl, ethyl, propyl, isopropyl, isobutyl and n-butyl substituted independently with one or more hydroxy groups.

As used herein, the term “C₃₋₇ cycloalkyl” refers to a non-aromatic cyclic hydrocarbon ring having from three to seven carbon atoms, which optionally includes a C₁₋₄ alkylene linker through which it may be attached. Exemplary “C₃₋₇ cycloalkyl” groups include, but are not limited to, cyclopropyl, cyclobutyl, cyclopentyl, cyclohexyl and cycloheptyl.

As used herein, the term “heterocyclic” or the term “heterocyclyl” refers to a three to twelve-membered non-aromatic ring being unsaturated or having one or more degrees of unsaturation containing one or more heteroatomic substitutions selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, tetrahydrofuran, pyran, 1,4-dioxane, 1,3-dioxane, piperidine, pyrrolidine, morpholine, tetrahydrothiopyran, tetrahydrothiophene, and the like.

As used herein, the term “aryl” refers to an optionally substituted benzene ring or to an optionally substituted benzene ring system fused to one or more optionally substituted benzene rings to form, for example, anthracene, phenanthrene, or napthalene ring systems. Exemplary optional substituents include lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. Examples of “aryl” groups include, but are not limited to, phenyl, 2-naphthyl, 1-naphthyl, biphenyl, as well as substituted derivatives thereof.

As used herein, the term “aralkyl” refers to an aryl or heteroaryl group, as defined herein including both unsubstituted and substituted versions thereof, attached through a lower alkylene linker, wherein lower alkylene is as defined herein. As used herein, the term “heteroaralkyl” is included within the scope of the term “aralkyl”. The term heteroaralkyl is defined as a heteroaryl group, as defined herein, attached through a lower alkylene linker, lower alkylene is as defined herein. Examples of “aralkyl”, including “heteroaralkyl”, include, but are not limited to, benzyl, phenylpropyl, 2-pyridinylmethyl, 4-pyridinylmethyl, 3-isoxazolylmethyl, 5-methyl-3-isoxazolylmethyl, and 2-imidazoyl ethyl.

As used herein, the term “arylamino” refers to an aryl or heteroaryl group, as defined herein, attached through an amino group —NR²—, wherein R² is as defined herein.

As used herein, the term “heteroaryl” refers to a monocyclic five to seven membered aromatic ring, or to a fused bicyclic aromatic ring system comprising two of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions and may be optionally substituted with up to three members selected from a group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. Examples of “heteroaryl” groups used herein include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof.

As used herein, the term “alkoxy” refers to the group RₐO—, where Rₐ is alkyl as defined above and the term “C₁₋₂ alkoxy” refers to the group RₐO—, where Rₐ is C₁₋₂ alkyl as defined above.

As used herein, the term “haloalkoxy” refers to the group RₐO—, where Rₐ is haloalkyl as defined above and the term “C₁₋₂ haloalkoxy” refers to the group RₐO—, where Rₐ is C₁₋₂ haloalkyl as defined above.

As used herein the term “aralkoxy” refers to the group R_{b}RₐO—, where Rₐ is alkylene and R_{b} is aryl, both as defined above.

As used herein the term “cyanoalkyl” refers to the group —RₐCN wherein Rₐ is C₁₋₃ alkylene as defined above. Exemplary “cyanoalkyl” groups useful in the present invention include, but are not limited to, cyanomethyl, cyanoethyl, and cyanopropyl.

As used herein, the term “aminosulfonyl” refers to the group —SO₂NH₂.

As used herein, the term “aroyl” refers to the group RₐC(O)—, where Rₐ is aryl as defined herein.

As used herein, the term “heteroaroyl” refers to the group RₐC(O)—, where Rₐ is heteroaryl as defined herein.

As used herein, the term “alkoxycarbonyl” refers to the group RₐOC(O)—, where Rₐ is alkyl as defined herein.

As used herein, the term “acyloxy” refers to the group RₐC(O)O—, where Rₐ is alkyl, cycloalkyl, or heterocyclyl as defined herein.

As used herein, the term “aroyloxy” refers to the group RₐC(O)O—, where Rₐ is aryl as defined herein.

As used herein, the term “heteroaroyloxy” refers to the group RₐC(O)O—, where Rₐ is heteroaryl as defined herein.

As used herein, the term “physiologically functional derivative” refers to any pharmaceutically acceptable derivative of a compound of formula I or II, for example, an ester or an amide, which upon administration to a mammal is capable of providing (directly or indirectly) a compound of formula I or II or an active metabolite thereof. Such derivatives are clear to those skilled in the art, without undue experimentation, and with reference to the teaching of Burger's Medicinal Chemistry and Drug Discovery, 5^{th} Edition, Vol. 1: Principles and Practice, which is incorporated herein by reference to the extent that it teaches physiologically functional derivatives.

As used herein, the term “solvate” refers to a complex of variable stoichiometry formed by a solute (in this invention, a compound of formula I or II) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water.

It is also noted that the compounds of formula I or II may form tautomers. It is understood that all tautomers and mixtures of tautomers of the compounds of the present invention, more specifically, the compounds of formula I or II are included within the scope of the compounds of the present invention.

The compounds of formulae I and II have the ability to crystallize in more than one form, a characteristic, which is known as polymorphism, and it is understood that such polymorphic forms (“polymorphs”) are within the scope of formulae I and II. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

Typically, the salts of the compounds of formulae I and II are pharmaceutically acceptable salts. Salts encompassed within the term “pharmaceutically acceptable salts” refer to non-toxic salts of the compounds of this invention. Salts of the compounds of formulae I and II may comprise acid addition salts derived from a nitrogen on a substituent in the compounds of formulae I and II. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate and ditosylate, triethiodide, trimethylammonium and valerate. Other salts, which are not pharmaceutically acceptable, may be useful in the preparation of compounds of this invention and these form a further aspect of the invention. Furthermore, such salt may be in anhydrous or hydrated form.

In one embodiment is the compound of formula I as a hydrochloride salt, preferably a monohydrochloride salt.

In one embodiment is the compound of formula II as a hydrochloride or ditosylate salt, preferably a ditosylate salt, more preferably the monohydrate of the ditosylate salt.

As recited above, a method of treating cancer is provided which includes administering a compound of formula I or a salt, solvate or physiologically functional derivative thereof and a compound of formula II or a salt, solvate or physiologically functional derivative thereof.

In one embodiment, the compound of formula I is a compound of formula I^{a}

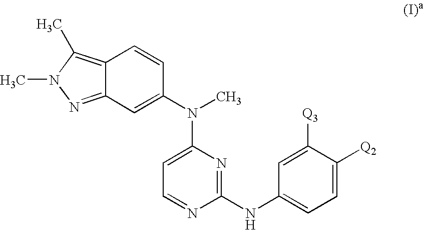

or a salt, solvate, or physiologically functional derivative thereof, wherein

Q₃ is A¹ when Q₂ is A² and Q₃ is A² when Q₂ is A¹;

wherein
A¹ is hydrogen, halogen, C₁₋₃ alkyl, and
A² is the group defined by -(Z)ₘ-(Z¹)-(Z²), wherein
   Z is CH₂ and m is 0, 1, 2, or 3;
   Z¹ is S(O)₂, S(O), or C(O); and
   Z² is C₁₋₄ alkyl, or NR³R⁴; and
R³ and R⁴ are each independently selected from hydrogen, or C₁₋₄ alkyl.

In a preferred embodiment, the compound of formula I is a compound of formula I^{b}

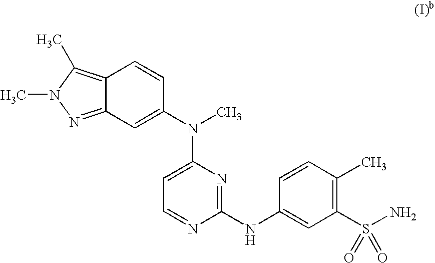

or a salt, solvate or physiological functional derivative thereof.

In another preferred embodiment, the compound of formula I is a compound of formula I^{c}

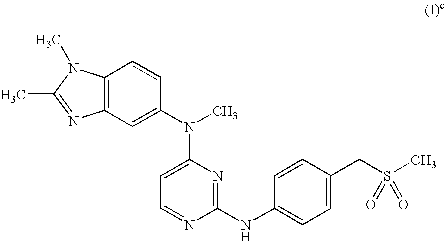

or salt, solvate or physiologically functional derivative thereof.

In another embodiment, the compound of formula II is a compound of formula II^{a}

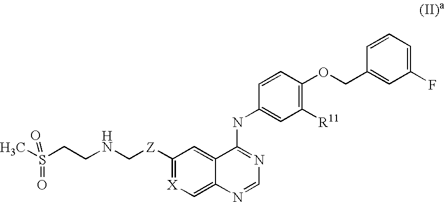

or a salt, solvate or physiologically functional derivative thereof, wherein R¹¹ is —Cl or —Br, X is CH, N, or CF, and Z is thiazole or furan.

In another preferred embodiment, the compound of formula II is a compound of formula II^{b}

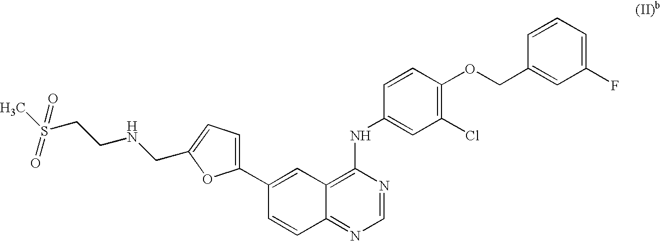

or a salt, solvate or physiologically functional derivative thereof.

In another preferred embodiment, the compound of formula II is a compound of formula II^{c}

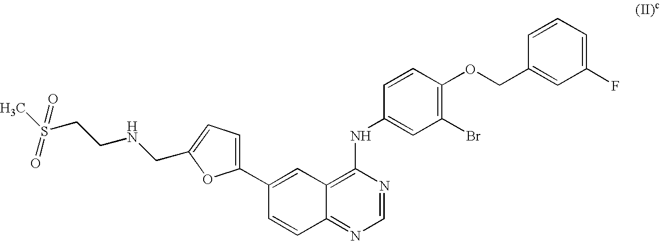

or a salt, solvate or physiologically functional derivative thereof.

In another preferred embodiment, the compound of formula II is a compound of formula II^{d}

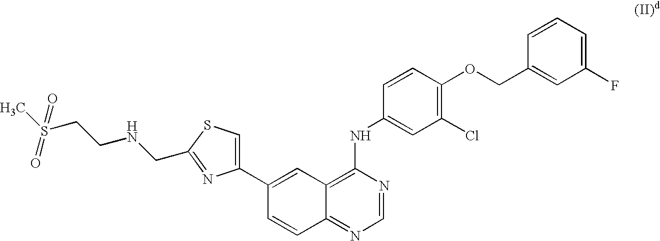

or a salt, solvate or physiologically functional derivative thereof.

In a most preferred embodiment the compound of formula I is a compound of formula I^{b} or a salt, solvate or physiologically functional derivative thereof, and the compound of formula II is a compound of formula II^{b} or a salt, solvate or physiologically functional derivative thereof.

In a most preferred embodiment the compound of formula I is a monohydrochloride salt of the compound of formula I^{b}, and the compound of formula II is a monohydrate ditosylate salt of the compound of the formula II^{b}.

In another most preferred embodiment the compound of formula I is a monohydrochloride salt of a compound of formula I^{b}, and the compound of formula II is an anhydrous ditosylate salt of a compound of the formula II^{b}.

While the preferred groups for each variable have generally been listed above separately for each variable, preferred compounds of this invention include those in which several of each variable in formulae (I) and (II) are selected from the preferred, more preferred, or most preferred groups for each variable. Therefore, this invention is intended to include all combinations of preferred, more preferred, and most preferred groups.

In the method of this invention, the compound of formula I and the compound of formula II may be employed in combination concomitantly or sequentially in any therapeutically appropriate combination. The compounds may be employed in combination in accordance with the invention by administration concomitantly in (1) a unitary pharmaceutical composition including both compounds or (2) separate pharmaceutical compositions each including one of the compounds. Alternatively, the compounds may be administered separately in a sequential manner wherein one is administered first and the other second or vice versa. Such sequential administration may be close in time or remote in time.

The cancer treatment method of the present invention may also include administration of at least one additional cancer treatment therapy in combination concomitantly or sequentially in any therapeutically appropriate combination with the combinations of the present invention. The additional cancer treatment therapy may include radiation therapy, surgical therapy and/or at least one additional chemotherapeutic therapy including administration of at least one additional anti-neoplastic agent.

In a preferred embodiment, the cancer treated by the method of the invention is breast, non-small cell lung, prostate, colorectal, renal, or bladder cancer. In another preferred embodiment, the cancer treated by the method of the invention is mesothelioma, hepatobiliary cancer, multiple myeloma, sarcoma, or leukemia.

While it is possible that, for use in therapy, the compound of formula I or the compound of formula II may be administered as the raw chemical, it is possible to present the active ingredient as a pharmaceutical composition. As indicated above, such elements of the pharmaceutical combination utilized may be presented in separate pharmaceutical compositions or formulated together in one pharmaceutical formulation. Accordingly, the invention further provides a combination of pharmaceutical compositions one of which includes a compound of the formula I and one or more pharmaceutically acceptable carriers, diluents, or excipients and a pharmaceutical composition containing a compound of the formula II and one or more pharmaceutically acceptable carriers, diluents, or excipients.

Alternatively, a pharmaceutical composition is provided which includes a compound of the formula I, a compound of the formula II, and one or more pharmaceutically acceptable carriers, diluents, or excipients. The compound of formula I and the compound of formula II are as described above and may be utilized in any of the combinations described above in the method of treating cancer of the present invention. A preferred composition may further comprise the preferred compounds, as described above, and one or more pharmaceutically acceptable carriers, diluents, or excipients.

The carrier(s), diluent(s) or excipient(s) must be acceptable in the sense of being compatible with the other ingredients of the formulation and not deleterious to the recipient thereof. According to another aspect of the invention there is also provided a process for the preparation of a pharmaceutical formulation including admixing a compound of the formula I, and a compound of the formula II, individually or together, with one or more pharmaceutically acceptable carriers, diluents or excipients.

The components of the pharmaceutical compositions of the present invention, may be formulated for administration by any route, and the appropriate route will depend on the specific cancer being treated as well as the subjects to be treated. Suitable pharmaceutical formulations include those for oral, rectal, nasal, topical (including buccal, sub-lingual, and transdermal), vaginal or parenteral (including intramuscular, sub-cutaneous, intravenous, and directly into the affected tissue) administration or in a form suitable for administration by inhalation or insufflation. The formulations may, where appropriate, be conveniently presented in discrete dosage units and may be prepared by any of the methods well know in the pharmacy art.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agents can also be present.

Capsules can be made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methylcellulose, agar, bentonite, xanthan gum and the like. Tablets can be formulated, for example, by preparing a powder mixture, granulating or slugging, adding a lubricant and disintegrant and pressing into tablets. A powder mixture can be prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture then can be compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The components of the pharmaceutical compositions of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The components of the pharmaceutical compositions of the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouthwashes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

A therapeutically effective amount of the components of the pharmaceutical compositions of the present invention will depend on a number of factors including, but not limited to, the age and weight of the mammal, the precise disorder requiring treatment and its severity, the nature of the formulation, and the route of administration, and will ultimately be at the discretion of the attendant physician or veterinarian. Typically, the components of the pharmaceutical compositions of the present invention will be given for treatment in the range of 0.1 to 100 mg/kg body weight of recipient (mammal) per day and more usually in the range of 1 to 10 mg/kg body weight per day. Acceptable daily dosages, may be from about 0.1 to about 1000 mg/day, and preferably from about 0.1 to about 100 mg/day.

The pharmaceutical compositions, including compounds of formula I and compounds of formula II, described above, are useful in therapy and in the preparation of medicaments for treating cancer in a mammal.

In one embodiment, the mammal in the methods and uses of the present invention is a human.

The following examples are intended for illustration only and are not intended to limit the scope of the invention in any way. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
i.v. (intravenous); Hz (Hertz);
MHz (megahertz); mol (moles);
mmol (millimoles); RT (room temperature);
min (minutes); h (hours);
mp (melting point); Tᵣ (retention time);
TLC (thin layer chromatography); RP (reverse phase);
MeOH (methanol); i-PrOH (isopropanol);
TEA (triethylamine); TFA (trifluoroacetic acid);
TFAA (trifluoroacetic anhydride); THF (tetrahydrofuran);
DMSO (dimethylsulfoxide); EtOAc (ethyl acetate);
DME (1,2-dimethoxyethane); DCM (dichloromethane);
DCE (dichloroethane);
DMF (N,N-dimethylformamide);
DMPU (N,N′-dimethylpropyleneurea); CDI (1,1-carbonyldiimidazole);
IBCF (isobutyl chloroformate); HOAc (acetic acid);
HOSu (N-hydroxysuccinimide);
HOBT (1-hydroxybenzotriazole);
mCPBA (meta-chloroperbenzoic acid); Et (ethyl);
EDC (ethylcarbodiimide hydrochloride); BOC (tert-butyloxycarbonyl);
FMOC (9-fluorenylmethoxycarbonyl);
DCC (dicyclohexylcarbodiimide);
CBZ (benzyloxycarbonyl); Ac (acetyl);
atm (atmosphere); TMSE (2-(trimethylsilyl)ethyl);
TMS (trimethylsilyl); TIPS (triisopropylsilyl);
TBS (t-butyldimethylsilyl); OMe (methoxy);
DMAP (4-dimethylaminopyridine); Me (methyl);
HPLC (high pressure liquid chromatography);
BOP (bis(2-oxo-3-oxazolidinyl)phosphinic chloride);
TBAF (tetra-n-butylammonium fluoride); tBu (tert-butyl).

All references to ether are to diethyl ether; brine refers to a saturated aqueous solution of NaCl. Unless otherwise indicated, all temperatures are expressed in ° C. (degrees Centigrade). All reactions conducted under an inert atmosphere at room temperature unless otherwise noted.

For the compounds of the formula I, ¹H NMR spectra were recorded on a Varian VXR-300, a Varian Unity-300, a Varian Unity-400 instrument, or a General Electric QE-300. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APIiii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a MeI-Temp II apparatus and are uncorrected.

For the compounds of the formula II ¹H NMR spectra were obtained at 500 MHz on a Bruker AMX500 spectrophotometer, on a Bruker spectrophotometer at 300 MHz, on a Bruker AC250 or Bruker AM250 spectrophotometer at 250 MHz and on a Varian Unity Plus NMR spectrophotometer at 300 or 400 MHz. J values are given in Hz. Mass spectra were obtained on one of the following machines: VG Micromass Platform (electrospray positive or negative), HP5989A Engine (thermospray positive) or Finnigan-MAT LCQ (ion trap) mass spectrometer. Analytical thin layer chromatography (tlc) was used to verify the purity of some intermediates which could not be isolated or which were too unstable for full characterization, and to follow the progress of reactions. Unless otherwise stated, this was done using silica gel (Merck Silica Gel 60 F254).

The free base and HCl salts of the compound of formula (I) may be prepared according to the procedures of the International Patent Application No. PCT/US01/49367, filed Dec. 19, 2001, and published as WO 02/059110 on Aug. 1, 2002, and International Patent Application No. PCT/US03/019211, filed Jun. 17, 2003 and published as WO 03/106416 on Dec. 24, 2003. Such application is incorporated herein by reference to the extent it teaches the preparation of the compounds of formula (I) and salts thereof. Some of such procedures are recited again herein as well as additional variations and procedures.

The free base, HCl salts, and ditosylate salts of the compound of formula (II) may be prepared according to the procedures of the International Patent Application No. PCT/EP99/00048, filed Jan. 8, 1999, and published as WO 99/35146 on Jul. 15, 1999. Such application is incorporated herein by reference to the extent it teaches the preparation of the compounds of formula (II) and salts thereof. Some of such procedures are recited again herein as well as additional variations and procedures.

The following examples describe the syntheses of intermediates particularly useful in the synthesis of compounds of formula (I):

### Intermediate Example 1

### Preparation of 2,3-dimethyl-6-nitro-2H-indazole

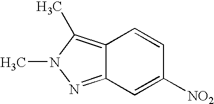

### Procedure 1:

To a stirred solution of 18.5 g (0.11 mol) of 3-methyl-6-nitro-1H-indazole in 350 ml acetone, at room temperature, was added 20 g (0.14 mol) of trimethyloxonium tetrafluoroborate. After the solution was allowed to stir under argon for 3 hours, the solvent was removed under reduced pressure. To the resulting solid was added saturated aqueous NaHCO₃ (600 mL) and a 4:1 mixture of chloroform-isopropanol (200 ml), the mixture was agitated and the layers were separated. The aqueous phase was washed with additional chloroform: isopropanol (4×200 mL) and the combined organic phase was dried (Na₂SO₄). Filtration and removal of solvent gave a tan solid. The solid was washed with ether (200 mL) to afford 2,3-dimethyl-6-nitro-2H-indazole as a yellow solid (15.85 g, 73%). ¹H NMR (300 MHz, DMSO-d₆) δ 8.51 (s, 1H), 7.94 (d, J=9.1 Hz, 1H), 7.73 (d, J=8.9 Hz, 1H), 4.14 (s, 3H), 2.67 (s, 3H). MS (ES+, m/z) 192 (M+H).

### Procedure 2:

Trimethyl orthoformate (11 mmol, 1.17 g) was added over a 2 min period to a solution of boron trifluoride etherate (12.5 mmol, 1.77 g in methylene chloride (2.0 mL) which had been cooled to −30° C. The mixture was warmed to 0° C. for 15 min and was then cooled to −70° C. The nitro indazole (10 mmol, 1.77 g) was slurried in methylene chloride (30 mL) and was added all at once to the cooled mixture. The mixture was stirred at −70° C. for 15 min and at ambient temperature for 17 h. After 17 h the mixture was red and heterogeneous. The reaction mixture was quenched with saturated sodium bicarbonate solution (20 mL) and the organic layer separated. The aqueous layer was extracted with methylene chloride (30 mL). The methylene chloride layers were combined and extracted with water (30 mL). The methylene chloride layer was distilled under reduced pressure until ˜10 mL remained. Propanol (10 mL) was added and the remainder of the methylene chloride removed under reduced pressure, resulting in a yellow slurry. The product was isolated by filtration to give 2,3-dimethyl-6-nitro-2H-indazole (65%, 7 mmol, 1.25 g) as a light yellow powder. ¹H NMR (300 MHz, DMSO-d₆) δ 8.51 (s, 1H), 7.94 (d, J=9.1 Hz, 1H), 7.73 (d, J=8.9 Hz, 1H), 4.14 (s, 3H), 2.67 (s, 3H). MS (ES+, m/z) 192 (M+H).

### Procedure 3:

In a 25 ml round bottom flask 3-methyl-6-nitroindazole (7.27 mmol, 1.28 g) was dissolved with stirring in DMSO (4.0 mL) and was treated with concentrated sulfuric acid (7.27 mmol, 0.73 g) to yield a thick slurry. The slurry was treated with dimethyl sulfate (21.1 mmol, 2.66 g). The mixture was heated under nitrogen at 50° C. for 72 h. After 72 h a thick yellow slurry was obtained. The slurry was cooled and was slowly treated with saturated sodium bicarbonate solution (10 mL). The mixture was extracted with methylene chloride (2×20 mL). The methylene chloride layers were combined and back extracted with water (20 mL). The methylene chloride layer was treated with propanol (10 mL) and the methylene chloride was removed by distillation under reduced pressure. The solid was isolated by filtration and the yellow solid washed with heptane (5 mL) and air-dried. The 2,3-dimethyl-6-nitro-2H-indazole product (70%, 0.97 g) was obtained as a light yellow solid. ¹H NMR (300 MHz, DMSO-d₆) δ 8.51 (s, 1H), 7.94 (d, J=9.1 Hz, 1H), 7.73 (d, J=8.9 Hz, 1H), 4.14 (s, 3H), 2.67 (s, 3H). MS (ES+, m/z) 192 (M+H).

### Procedure 4:

Into a 250 mL 3-necked round bottom flask was placed 3-methyl-6-nitro-1H-indazole sulfuric acid salt (5.0 g, 18.2 mmol) and methylene chloride (25 mL). The mixture was stirred at 25° C. and was treated with DMSO (5 mL). Dimethyl sulfate (6.7 g, 5.0 mL, 53.0 mmol) was added via syringe and the reaction was heated at reflux in a 70° C. bath. After 7 h HPLC analysis showed 9% starting material. At this point heating was stopped and the workup begun. Saturated sodium bicarbonate solution (35 mL) was added to the reaction mixture at RT. The layers were allowed to separate and the aqueous layer was extracted with methylene chloride (25 mL). The methylene chloride layers were combined and washed with water (2×25 mL). The methylene chloride layer was distilled under reduced pressure until half the volume was removed. Propanol (25 mL) was added and distillation under reduced pressure was continued until all the methylene chloride had been removed. This yielded a yellow slurry, which was allowed to stir at 25° C. for 1 h. The product was isolated via filtration and the resulting yellow solid was washed with heptane (10 mL). This yielded 2,3-dimethyl-6-nitro-2H-indazole (70%, 2.43 g) as a yellow solid. ¹H NMR (300 MHz, DMSO-d₆) δ 8.51 (s, 1H), 7.94 (d, J=9.1 Hz, 1H), 7.73 (d, J=8.9 Hz, 1H), 4.14 (s, 3H), 2.67 (s, 3H). MS (ES+, m/z) 192 (M+H).

### Intermediate Example 2

### Preparation of 2,3-dimethyl-6-amino-2H-indazole

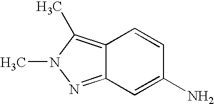

### Procedure 1:

To a stirred solution of 2,3-dimethyl-6-nitro-2H-indazole (1.13 g) in 2-methoxyethyl ether (12 ml), at 0° C., was added a solution of 4.48 g of tin(II) chloride in 8.9 ml of concentrated HCl dropwise over 5 min. After the addition was complete, the ice bath was removed and the solution was allowed to stir for an additional 30 min. Approximately 40 ml of diethyl ether was added to reaction, resulting in precipitate formation. The resulting precipitate was isolated by filtration and washed with diethyl ether, and afforded a yellow solid (1.1 g, 95%), the HCl salt 2,3-dimethyl-2H-indazol-6-amine. ¹H NMR (300 MHz, DMSO-d₆) δ 7.77 (d, J=8.9 Hz, 1H), 7.18 (s, 1H), 7.88 (m, 1H), 4.04 (s, 3H), 2.61 (s, 3H). MS (ES+, m/z) 162 (M+H).

### Procedure 2:

A 2-L 3-necked round bottom flask was fitted with nitrogen inlet and outlet and with mechanical stirring. A moderate nitrogen flow was initiated and the reactor was charged with 10% Pd/C (50% water wet, 6.0 g). Stirring was initiated and the reactor was charged with methanol (750 mL) and the product of Intermediate Example 1 (50 g). Ammonium formate (82.54 g) was dissolved in water (120 mL). The water solution of ammonium formate was added to the reaction solution at an addition rate, which kept the reaction temperature at or between 25 and 30° C. The reaction was allowed to proceed at 25° C. After 6 h the reaction was judged to be finished based on HPLC analysis. The mixture was filtered and the catalyst washed with methanol (50 mL). The methanol layers were combined and the solvent removed under reduced pressure. The residue was dissolved in water (200 mL) and was extracted with methylene chloride (3×250 mL). The methylene chloride layers were combined and solvent removed under vacuum to remove approximately half the solvent. Heptane (400 mL) was added and the vacuum distillation continued until approximately 300 mL reaction product slurry remained. The product was isolated by filtration and dried under vacuum at 50° C. for 4 h. to yield 2,3-dimethyl-6-amino-2H-indazole as the free base. (40.76 g, 96.7%). ¹H NMR (300 MHz, DMSO-d₆) δ 7.31 (d, J=8.9 Hz, 1H), 6.45 (d, J=8.9 Hz, 1H), 6.38 (s, 1H), 4.95 (s, br, 2H), 3.85 (s, 3H), 2.44 (s, 3H) MS (ES+, m/z) 162 (M+H).

### Intermediate Example 3

### Preparation of N-(2-chloropyrimidin-4-yl)-2,3-dimethyl-2H-indazol-6-amine

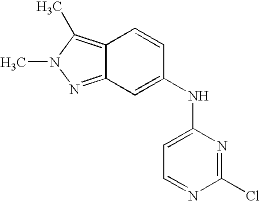

### Procedure 1

To a stirred solution of the product of Intermediate Example 2 (2.97 g, 0.015 mol) and NaHCO₃ (5.05 g, 0.06 mol) in THF (15 mL) and ethanol (60 mL) was added 2,4-dichloropyrimidine (6.70 g, 0.045 mol) at rt. After the reaction was stirred for four hours at 85° C., the suspension was cooled to rt., filtered and washed thoroughly with ethyl acetate. The filtrate was concentrated under reduced pressure, and the resulting solid was triturated with ethyl acetate to yield N-(2-chloropyrimidin-4-yl)-2,3-dimethyl-2H-indazol-6-amine (89%, 3.84 g). ¹H NMR (400 MHz, DMSO-d₆) δ 7.28 (d, J=9.0 Hz, 1H), 6.42 (d, J=8.8 Hz, 1H), 6.37 (s, 1H), 5.18 (br s, 1H), 3.84 (s, 3H), 2.43 (s, 3H). MS (ES+, m/z) 274 (M+H).

### Procedure 2

To a 1-L 3-necked flask equipped with air-driven mechanical stirrer, thermometer, and nitrogen inlet/outlet was charged a solution of the product of Intermediate Example 2 (32.89 g, 0.204 mol, 1.0 equiv) in 425 mL (13 volumes) of EtOH/THF (4/1), sodium bicarbonate (51.42 g, 0.612 mol, 3.0 equiv) and then 2,4-dichloropyrimidine (45.59 g, 0.306 mol, 1.5 equiv). The flask contents were heated to 75° C. and held at 74-76° C. for 6-7 hrs. The progress of the reaction was checked by HPLC (the product of Intermediate Example 2 <2%). The reaction contents were cooled to 20-25° C. over 30 min, and kept at 20-25° C. for 30 min. Then the reaction contents were further cooled to 10-12° C. over 30 min, and kept at that temperature for an additional 10 min. The contents were filtered and filter cake washed with EtOAc (2×100 mL, 3.0 volumes), and deionized water (514 mL, 15.6 volumes). The filter cake was then dried in a vacuum oven at 35° C. overnight to afford the desired product 44.75 g as a white solid (80.1%). ¹H NMR (400 MHz, DMSO-d₆) δ 7.28 (d, J=9.0 Hz, 1H), 6.42 (d, J=8.8 Hz, 1H), 6.37 (s, 1H), 5.18 (br s, 1H), 3.84 (s, 3H), 2.43 (s, 3H). MS (ES+, m/z) 274 (M+H).

### Intermediate Example 4

### Preparation of N-(2-chloropyrimidin-4-yl)-N,2,3-trimethyl-2H-indazol-6-amine

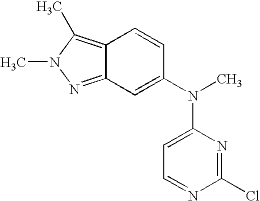

Procedure 1

To a stirred solution of the product of Intermediate Example 3 (7.37 g) in DMF (50 ml) was added Cs₂CO₃ (7.44 g, 2 eqv.) and iodomethane (1.84 ml, 1.1 eqv.) at room temperature. The mixture was stirred at rt overnight. The reaction mixture was then poured into an ice-water bath, and the precipitate was collected via filtration and washed with water. The precipitate was air-dried to afford N-(2-chloropyrimidin-4-yl)-N,2,3-trimethyl-2H-indazol-6-amine as an off-white solid (6.43 g, 83%). ¹H NMR (400 MHz, DMSO-d₆) δ 7.94 (d, J=6.0 Hz, 1H), 7.80 (d, J=7.0 Hz, 1H), 7.50 (d, J=1.0 Hz, 1H), 6.88 (m, 1H), 6.24 (d, J=6.2 Hz, 1H), 4.06 (s, 3H), 3.42 (s, 3H), 2.62 (s, 3H). MS (ES+, m/z) 288 (M+H).

### Procedure 2

A 3 L 3-necked flask equipped with air-driven mechanical stirrer, thermometer, addition funnel and nitrogen inlet/outlet was charged with DMF (272 mL, 5 volumes) and the product of Intermediate Example 3 (54.4 g, 0.20 mol, 1.0 equiv) with stirring. The reaction mixture was further charged with cesium carbonate (194.5 g, 0.60 mol, 3.0 equiv) while maintaining the reaction temperature between 20˜25° C. The reaction mixture was stirred at 20˜25° C. for 10 minutes. Iodomethane (45.1 g, 0.32 mol, 1.6 equiv) was charged over ˜10 minutes while maintaining the temperature 20˜30° C. The reaction mixture was stirred at 20˜30° C. (Typically, the reaction is complete in 1˜2 hours). Deionized H₂O (925 mL, 17 volumes) was added over ˜30 minutes while maintaining the temperature at 25˜40° C. The reaction mixture was stirred at 20˜25° C. for 40 minutes. The product was isolated by filtration and then the filter cake washed with H₂O/DMF (6:1, 252 mL, 4.6 volumes). The wet cake was dried under vacuum at 40˜45° C. and N-(2-chloropyrimidin-4-yl)-N,2,3-trimethyl-2H-indazol-6-amine (51.7 g, 90.4%) was isolated as a yellow solid. ¹H NMR (400 MHz, DMSO-d₆) δ 7.94 (d, J=6.0 Hz, 1H), 7.80 (d, J=7.0 Hz, 1H), 7.50 (d, J=1.0 Hz, 1H), 6.88 (m, 1H), 6.24 (d, J=6.2 Hz, 1H), 4.06 (s, 3H), 3.42 (s, 3H), 2.62 (s, 3H). MS (ES+, m/z) 288 (M+H).

### Intermediate Example 5

### Preparation of 5-amino-2-methylbenzenesulfonamide

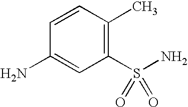

### Procedure 1

To a stirred solution of 2-methyl-5-nitrobenzenesulfonamide (4.6 g, 0.021 mol) in 2-methoxyethyl ether (43 mL), at 0° C., was added a solution of 16.1 g of tin(II) chloride in 32 mL of concentrated HCl dropwise over 15 min. After the addition was complete, the ice bath was removed and the solution was allowed to stir for an additional 30 min. Approximately 130 mL of diethyl ether was added to reaction. The mixture was stirred vigorously for 1 h. The mixture was basified with a solution of NaOH and NaHCO₃, and extracted with ethyl acetate (×3). The combined ethyl acetate layers were dried over anhydrous MgSO₄, filtered and concentrated to give crude product. Trituation of the crude product with methanol provided 2.4 g of pure 5-amino-2-methylbenzenesulfonamide as light brown solid. ¹H NMR (300 MHz, DMSO-d₆) δ 7.11-7.10 (m, 3H), 6.95 (d, J=8.1 Hz, 1H), 6.60 (dd, J=8.1 & 2.4 Hz, 1H), 5.24 (s, 2H), 2.36 (s, 3H). MS (ES+, m/z) 187 (M+H).

### Intermediate Example 6

### Preparation of 4-[(methylsulfonyl)methyl]aniline

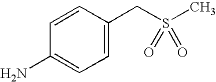

### Procedure 1

Combine 4-nitrobenzyl bromide (40 g, 0.185 mol) and sodium methanesulphinic acid (19.5 g, 1 eqv.) in ethanol (460 mL, ˜0.4M). The mixture was stirred and heated to 80° C. under reflux. After 3 hr the reaction mixture was cooled to rt and filtered to collected off-white solid. The solid was washed with EtOH twice and air-dried to provide 37 g of methyl 4-nitrobenzyl sulfone. ¹H NMR (300 MHz, DMSO-d₆) δ 8.27 (d, J=8.6 Hz, 2H), 7.69 (d, J=8.6 Hz, 2H), 4.71 (s, 2H), 2.96 (s, 3H). MS (ES+, m/z) 216 (M+H).

Combined methyl 4-nitrobenzyl sulfone (9.5 g, 0.044 mol) and 10% Pd/C (0.95 g, 0.1 w/w) in ethyl acetate (220 mL, ˜0.2M). The mixture was placed under Parr shaker with 40 psi of hydrogen. After ˜3 hr, the reaction mixture was poured into 50% of MeOH/EtOAc (400 mL) and stirred vigorously for 30 min. The mixture was filtered through a pad of celite and silica gel. The black material on top of the pad was removed and placed into 80% MeOH/EtOAc (200 mL) and stirred vigorously for 30 min. The mixture was again filtered through a pad of celite and silica gel. The process is repeated a couple times. Combined all filtrates. Evaporated and dried. Trituation with EtOAc provided pure 4-[(methylsulfonyl)methyl]aniline. ¹H NMR (300 MHz, DMSO-d₆) δ 7.03 (d, J=8.4 Hz, 2H), 6.54 (d, J=8.6 Hz, 2H), 5.20 (s, 2H), 4.20 (s, 2H), 2.79 (s, 3H). MS (ES+, m/z) 186 (M+H).

### Procedure 2

Charge a round bottom flask (1.0 L), equipped with magnetic stir bar and reflux condenser, with 4-nitrobenzyl bromide (40 g, 0.185 mol, 1.0 eq.), sodium methanesulphinic acid (21.7 g, 0.213 mol, 1.15 eq.) and ethanol (400 mL, 200 proof, 10 vol.). Stir and heat the mixture to 80° C. under reflux for 2 hours. Check the progress of the reaction by fast-HPLC (reaction is deemed complete when HPLC indicates 4-nitrobenzyl bromide <0.5%). Cool the mixture to room temperature. Filter and wash the cake with ethanol (40 mL). The wet cake (15 g, 46.2 mmol) was used for next step hydrogenation with out further dry.

Charge a 500 mL of hydrogenation flask with above wet cake methyl 4-nitrobenzyl sulfone (15 g, 46.2 mmol, used “as is”), 10% Pd/C (0.1 g, 1% w/w) and ethanol (120 mL, 200 proof) and water (40 mL). Swap the atmosphere of reactor with hydrogen (3 times). Shake the reactor under H₂ (65 psi) at room temperature for 30 minutes and at 50° C. for two hour. Check the progress of the reaction by HPLC (reaction is deemed complete when HPLC indicates methyl 4-nitrobenzyl sulfone <0.2%). Heat the mixture to 80° C. Filter the hot solution through a pad of celite (2.0 g) and rinse the pad with EtOH (10 mL). Transfer the filtrate into the crystallizing a round bottom flask (500 mL). Distil the slurry under house vacuum at 60° C. until a volume of 60 mL is left. Cool the slurry to 0° C. over for one hour. Isolate the crystals by vacuum filtration and wash the vessel and crystals with ethanol (10 mL). Dry the product under house vacuum at 50° C. to constant weight. Obtained off-white solid (7.3 g). The yield is 85% for combined two steps with 99% purity of product by HPLC.

### Intermediate Example 7

### Preparation of 4-[(isopropylsulfonyl)methyl]phenylamine

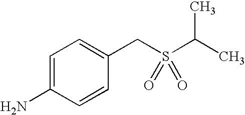

To a solution of 1-(bromomethyl)-4-nitrobenzene (3.0 g, 17.4 mmol) in ethanol (50 mL) was added sodium-2-thiopropoylate (2.7 g, 17.4 mmol). After 12 h the solvent was removed under reduced pressure, the remaining residue was diluted with EtOAc and filtered to remove the residual salts. The solvent was dried over MgSO₄ and removed under reduced pressure and the product was carried forward without further purification. Next the sulfide was diluted with CH₂Cl₂ (50 mL) and m-chloroperoxybenzoic acid (˜70%) (6.6 g, 38.4 mmol) was added in portions. The reaction was judged to be complete by tlc and the solvent was removed under reduced pressure. The remaining residue was diluted with EtOAc and washed with 1M NaOH (2×100 mL). The solvent was dried over MgSO₄ and removed under reduced pressure and the product was carried forward without further purification. Next the residue was diluted with glyme (8.0 mL) and a solution of SnCl₂ (13.8 g, 69 mmol) in HCl (8.0 mL) was added dropwise. The solution was allowed to stir for 2 h, and the reduction was judged to be complete by tlc. The reaction mixture was diluted with Et₂O, which resulted in the precipitation of the product as the HCl salt. The solids were collected and washed with Et₂O (2×100 mL), to afford pure aniline (˜2.4 g, 65%). ¹H NMR (300 MHz, d₆DMSO+NaHCO₃) δ 7.37 (d, J=8.4 Hz, 2H), 7.21 (d, J=8.4 Hz, 2H), 4.41 (s, 2H), 3.18-3.09 (m, 1H), 1.21 (d, J=6.9 Hz, 6H).

### Intermediate Example 8

### Preparation of 4-[2-(methylsulfonyl)ethyl]aniline

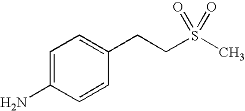

To a solution of 1-(bromoethyl)-4-nitrobenzene (3.0 g, 13.0 mmol) in ethanol (70 mL) was added Sodium thiomethoxide (1.0 g, 14.0 mmol). After 12 h the solvent was removed under reduced pressure, the remaining residue was diluted with EtOAc and filtered to remove the residual salts. The solvent was dried over MgSO4 and removed under reduced pressure and the product was carried forward without further purification. Next the sulfide was diluted with CH₂Cl₂ (100 mL) and m-chloroperoxybenzoic acid (˜70%) (8.2 g, 48.8 mmol) was added in portions. The reaction was judged to be complete by tlc and the solvent was removed under reduced pressure. The remaining residue was diluted with EtOAc and washed with 1M NaOH (2×100 mL). The solvent was dried over MgSO₄ and removed under reduced pressure and the product was carried forward without further purification. Next the residue was added to a slurry of Palladium on Carbon (10 mol %) in EtOAc (50 mL) in a Parr shaker vessel. The reaction was then place under 40 atm of Hydrogen gas. The solution was allowed to shake for 2 h, and the reduction was judged to be complete by tlc. The reaction mixture was filtered over a pad of celite and washed with EtOAc and the solvent was removed under reduced pressure to afford a crude solid. The mixture was recrystallized in hot EtOAc to afford the pure aniline (˜1.8 g, 69%). ¹H NMR (300 MHz, d₆DMSO+NaHCO₃) δ 6.93 (d, J=8.2 Hz, 2H), 6.87 (d, J=8.2 Hz, 2H), 5.09 (bs, 2H), 3.31-3.26 (m, 2H), 2.92 (s, 3H), 2.84-2.79 (m, 2H).

### Intermediate Example 9

### Preparation of 4-[1-(methylsulfonyl)ethyl]aniline

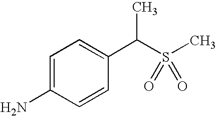

To a solution of 4-nitrophenylcarbonol (3.0 g, 17.9 mmol) and triethylamine (3.5 mL, 21.0 mmol) in CH₂Cl₂ (100 mL) was added methanesulfonylchloride (1.7 mL, 21.0 mmol) dropwise. The reaction was judged to be complete by tlc after 1 h and was quenched with saturated aqueous NaHCO3. The reaction mixture was diluted with EtOAc and the organic layer separated, dried over MgSO₄ and the solvent was removed under reduced pressure. The resulting residue was dissolved in ethanol (100 mL) and Sodium thiomethoxide (1.5 g, 21.0 mmol) was added in portions. After 12 h the solvent was removed under reduced pressure, the remaining residue was diluted with EtOAc and filtered to remove the residual salts. The solvent was dried over MgSO₄ and removed under reduced pressure and the product was carried forward without further purification. Next the sulfide was diluted with CH₂Cl₂ (100 mL) and m-chloroperoxybenzoic acid (˜70%) (10.8 g, 62 mmol) was added in portions. The reaction was judged to be complete by tlc and the solvent was removed under reduced pressure. The remaining residue was diluted with EtOAc and washed with 1M NaOH (2×100 mL). The solvent was dried over MgSO₄ and removed under reduced pressure and the product was carried forward without further purification. Next the residue was added to a slurry of Palladium on Carbon (10 mol %) in EtOAc (50 mL) in a Parr shaker vessel. The reaction was then place under 40 atm of Hydrogen gas. The solution was allowed to shake for 2 h, and the reduction was judged to be complete by tlc. The reaction mixture was filtered over a pad of celite and washed with EtOAc and the solvent was removed under reduced pressure to afford a crude solid. The mixture was recrystallized in hot EtOAc to afford the pure aniline (˜2.0 g, 57%). ¹H NMR (300 MHz, d₆DMSO+NaHCO₃) δ 7.06 (d, J=8.5 Hz, 2H), 6.53 (d, J=8.5 Hz, 2H), 5.21 (s, 2H), 4.23 (q, J=7.1 Hz, 1H), 2.70 (s, 3H), 1.21 (d, J=7.1 Hz, 3H).

### Intermediate Example 10

### Preparation of 4-[1-methyl-1-(methylsulfonyl)ethyl]aniline

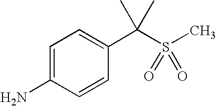

To a stirred solution of t-butoxide (5.76 g, 0.051 mol) in THF was added methyl 4-nitrobenzyl sulfone (5 g, 0.023 mol) followed by iodomethane (2.89 ml, 0.046 mol). The mixture was stirred at rt for 1 hr. Additional t-butoxide (2.9 g) and iodomethane (0.5 ml) were added. The mixture was stirred at rt for additional 1 hr. The mixture was diluted with EtOAc and acidified with 6N HCl. The mixture was extracted with ethyl acetate (×3). The combined ethyl acetate layers were dried over anhydrous MgSO4, filtered and evaporated. The solid was trituated with ethanol to give pure 1-[1-methyl-1-(methylsulfonyl)ethyl]-4-nitrobenzene.

To a stirred solution of 1-[1-methyl-1-(methylsulfonyl)ethyl]-4-nitrobenzene (3.32 g, 0.014 mol) in 2-methoxyethyl ether (70 mL), at 0° C., was added a solution of 10.35 g of tin(II) chloride in 20.5 mL of concentrated HCl dropwise over 15 min. After the addition was complete, the ice bath was removed and the solution was allowed to stir for an additional 30 min. Approximately 70 mL of diethyl ether was added to reaction. The mixture was stirred vigorously for 1 h. Precipitate was formed and was collected via filtration. The solid was dissolved in CH₂Cl₂ and washed with 1N NaOH. The mixture was extracted with CH₂Cl₂ (×3). The combined CH₂Cl₂ layers were dried over anhydrous MgSO₄, filtered and evaporated to give 4-[1-methyl-1-(methylsulfonyl)ethyl]aniline as an off white solid. ¹H NMR (300 MHz, DMSO-d₆) δ 7.21 (d, J=8.6 Hz, 2H), 6.55 (d, J=8.6 Hz, 2H), 5.23 (s, 2H), 2.58 (s, 3H), 1.64 (s, 6H).

### Example 1

### 5-({4-[(2,3-dimethyl-2H-indazol-6-yl)(methyl)amino]pyrimidin-2-yl}amino)-2-methylbenzenesulfonamide

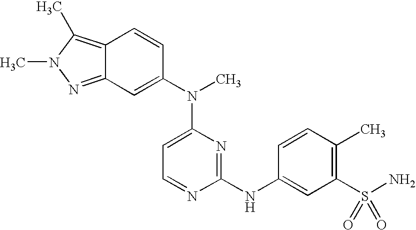

### Procedure 1

To a solution of Intermediate Example 4 (200 mg, 0.695 mmol) and 5-amino-2-methylbenzenesulfonamide (129.4 mg, 0.695 mmol) in isopropanol (6 ml) was added 4 drops of conc. HCl. The mixture was heated to reflux overnight. The mixture was cooled to rt and diluted with ether (6 ml). Precipitate was collected via filtration and washed with ether. The hydrochloride salt of 5-({4-[(2,3-dimethyl-2H-indazol-6-yl)(methyl)amino]-pyrimidin-2-yl}amino)-2-methylbenzenesulfonamide was isolated as an off-white solid. ¹H NMR (400 MHz, d₆DMSO+NaHCO₃) δ 9.50 (br s, 1H), 8.55 (br s, 1H), 7.81 (d, J=6.2 Hz, 1H), 7.75 (d, J=8.7 Hz, 1H), 7.69 (m, 1H), 7.43 (s, 1H), 7.23 (s, 2H), 7.15 (d, J=8.4 Hz, 1H), 6.86 (m, 1H), 5.74 (d, J=6.1 Hz, 1H), 4.04 (s, 3H), 3.48 (s, 3H), 2.61 (s, 3H), 2.48 (s, 3H). MS (ES+, m/z) 438 (M+H).

### Procedure 2

A 250-mL 3-necked flask equipped with a magnetic stir bar, thermometer, reflux condenser, and nitrogen inlet/outlet was charged with ethanol (60 mL, 10 volumes), the product of Intermediate Example 4 (6.00 g, 20.85 mmol, 1.0 equiv) and 5-amino-2-methylbenzenesulfonamide (4.00 g, 21.48 mmol, 1.03 equiv) with stirring. The reaction mixture was heated to 70° C. After stirring the reaction mixture at 68-72° C. for 3 hrs, 4M HCl in dioxane (0.11 mL, 0.44 mmol, 0.02 equiv) was charged over ca. 2 min. The reaction mixture was stirred at 68-72° C. until <1.5% by area of the starting product of Intermediate Example 4 was remaining by HPLC analysis (Typically, this reaction is complete in >8 hrs). The reaction mixture was cooled to 20° C. over ca. 30 min and stirred at 20-22° C. for 40 min. The product was then isolated by filtration and the filter cake washed with ethanol (20 mL, 3.3 volumes). The wet cake was dried under vacuum at 45-50° C. The monohydrochloride salt of 5-({4-[(2,3-dimethyl-2H-indazol-6-yl)(methyl)amino]-pyrimidin-2-yl}amino)-2-methylbenzenesulfonamide (9.52 g, 96.4%) was isolated as a white solid. ¹H NMR (400 MHz, d₆DMSO+NaHCO₃) δ 9.50 (br s, 1H), 8.55 (br s, 1H), 7.81 (d, J=6.2 Hz, 1H), 7.75 (d, J=8.7 Hz, 1H), 7.69 (m, 1H), 7.43 (s, 1H), 7.23 (s, 2H), 7.15 (d, J=8.4 Hz, 1H), 6.86 (m, 1H), 5.74 (d, J=6.1 Hz, 1H), 4.04 (s, 3H), 3.48 (s, 3H), 2.61 (s, 3H), 2.48 (s, 3H). MS (ES+, m/z) 438 (M+H).

### Procedure 3:

To a stirred suspension of the product of Intermediate Example 4 (1.1 g, 3.8 mmol) in 14 mL of MeOH, was added 5-amino-2-methylbenzenesulfonamide (0.78 g, 4.2 mmol, 1.1 equiv) at room temperature. The reaction mixture was heated at reflux for 3 h, then 4 M HCl in 1,4-dioxane (19 μL, 0.076 mmol) was added in one portion. After 4 h, the suspension was cooled to room temperature, and filtered. The resulting solid was washed with 10 mL of MeOH and dried in vacuo to yield 1.3 g (72%) of 5-({4-[(2,3-dimethyl-2H-indazol-6-yl)methylamino]-2-pyrimidinyl}amino)-2-methyl benzenesulfonamide monohydrochloride as a white solid. ¹H NMR (DMSO-d₆, 400 MHz) δ 10.95 (s, 1H), 8.36 (s, 1H), 7.86 (d, J=8.8 Hz, 2H), 7.64-7.59 (m, 2H), 7.40 (m, 3H), 6.93 (dd, J=8.8, 2.0 Hz, 1H), 5.92 (s, 1H), 4.08 (s, 3H), 3.57 (s, 3H), 2.65 (s, 3H), 2.56 (s, 3H).

### Procedure 4

To a stirred suspension of the product of Intermediate Example 4 (1.1 g, 3.7 mmol) in 10 mL of THF, was added 5-amino-2-methylbenzenesulfonamide (0.70 g, 3.8 mmol, 1.0 equiv) at room temperature. The reaction mixture was heated at reflux for 3 h, then 4 M HCl in 1,4-dioxane (18 μL, 0.072 mmol) was added in one portion. After 5 h, the suspension was cooled to room temperature, and filtered. The resulting solid was washed with 16 mL of THF and dried in the air to yield 1.6 g (92%) of 5-({4-[(2,3-dimethyl-2H-indazol-6-yl)methylamino]-2-pyrimidinyl}amino)-2-methylbenzene sulfonamide monohydrochloride as a light yellow solid.

### Procedure 5

To a stirred suspension of the product of Intermediate Example 4 (1.0 g, 3.6 mmol) in 10 mL of CH₃CN, was added 5-amino-2-methylbenzenesulfonamide (0.70 g, 3.8 mmol, 1.0 equiv) at room temperature. The reaction mixture was heated at reflux for 3 h, then 4 M HCl in 1,4-dioxane (18 μL, 0.076 mmol) was added in one portion. After 20 h, the suspension was cooled to room temperature, and filtered. The resulting solid was washed with 10 mL of CH₃CN and dried in the air to yield 1.3 g (73%) of 5-({4-[(2,3-dimethyl-2H-indazol-6-yl)methylamino]-2-pyrimidinyl}amino)-2-methyl benzenesulfonamide monohydrochloride as an off-white solid.

### Procedure 6

### Preparation of 5-({4-[(2,3-dimethyl-2H-indazol-6-yl)(methyl)amino]pyrimidin-2-yl}amino)-2-methylbenzenesulfonamide methanesulfonic acid salt

In a 250 mL flask the product of Example 1, procedure 1, (1.0 g, 2.29 mmol) was slurried in water (19 mL). Methanesulfonic acid (0.231 g, 2.4 mmol) was added all at once and the mixture was heated to reflux for 5 min. The mixture was cooled to 0° C. over a 1 hour period and was then isolated by filtration and air dried. 5-({4-[(2,3-Dimethyl-2H-indazol-6-yl)(methyl)amino]pyrimidin-2-yl}amino)-2-methylbenzenesulfonamide methanesulfonic acid salt (1.03 g, 84%) was obtained as a white solid. mp=247-248° C.

### Procedure 7:

### Preparation of 5-({4-[(2,3-dimethyl-2H-indazol-6-yl)methylamino]-2-pyrimidinyl}amino)-2-methylbenzenesulfonamide monohydrochloride monohydrate

To a round bottom flask, was added 2.6 g of the monohydrochloride salt of Example 1, procedure 1, any form. Then added was 39 mL of isopropanol (15 volumes). The mixture was heated to 75 deg C. in an oil bath, then 14 mL of 0.05N aqueous HCl (5.4 volumes) was added. The clear solution was cooled to 65 deg C., then seeded with the monohydrate of the monohydrochloride salt of Example 1, procedure 1 (0.05-0.1 wt %). The cloudy solution was stirred at 65 deg C. for 60 minutes, then cooled to 0 deg C. at ˜0.25-0.5 deg C./min. The resulting white solid was filtered and dried to constant weight under vacuum at RT to give 88% yield of 5-({4-[(2,3-dimethyl-2H-indazol-6-yl)methylamino]-2-pyrimidinyl}amino)-2-methylbenzene sulfonamide monohydrochloride monohydrate.

Example 2 was prepared according to the general procedure set forth above in Example 1 using Intermediate Example 4 and the appropriate aniline. The appropriate anilines were prepared using procedures similarly described for Intermediate Examples 5-10.

### Example 2

### N⁴-(2,3-dimethyl-2H-indazol-6-yl)-N⁴-methyl-N²-{4-[(methylsulfonyl)methyl]phenyl}pyrimidine-2,4-diamine

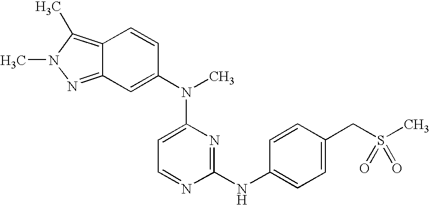

¹H NMR (300 MHz, d₆DMSO+NaHCO₃) δ 9.37 (bs, 1H), 7.88 (d, J=6.1 Hz, 1H), 7.78 (m, 3H), 7.47 (s, 1H), 7.22 (d, J=8.5 Hz, 2H), 6.91 (dd, J=8.8, 1.5 Hz, 1H), 5.84 (d, J=6.1 Hz, 1H), 4.37 (s, 2H), 4.09 (s, 3H), 3.51 (s, 3H), 2.88 (s, 3H), 2.65 (s, 3H). MS (ES+, m/z) 437 (M+H), 435 (M−H).

### Example 3

### Monohydrate ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (monohydrate ditosylate salt of compound of formula (II))

### 1(a) Preparation of N-{3-Chloro-4-[(3-fluorobenzyl)oxy]phenyl}-6-[5-({[2-(methane sulphonyl)ethyl]amino}methyl)-2-furyl]-4-quinazolinamine (free base of compound of formula (II))

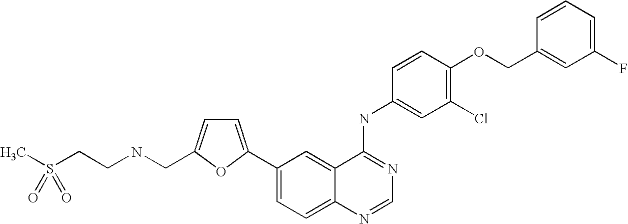

The title compound was prepared according to Procedure D of International Applications WO 02/02552: p. 16, line 19 to p. 17, line 3 and WO 99/35146: p. 56, lines 20-32 and Example 29p. 100, lines 18-29, from 5-(4-{3-chloro-4-(3-fluorobenzyloxy)-anilino}-6-quinazolinyl)-furan-2-carbaldehyde (0.6 equiv) and 2-methanesulphonyl-ethylamine (1 equiv). ¹H NMR 400 MHz (DMSO-d₆) 9.60 (bs, 1H); 9.32 (bs, 1H); 8.82 (bs, 1H); 8.34 (d, 1H); 8.0 (s, 1H); 7.88 (d, 1H); 7.74 (d, 1H); 7.45 (m, 1H); 7.34-7.23 (m, 4H); 7.17 (m, 1H); 6.83 (d, 1H); 5.27 (s, 2H); 4.42 (s, 2H); 3.59 (m, 2H); 3.40 (m, 2H, obscured by waterpeak); 3.12 (s, 3H); MS m/z 581 (M+H⁺).

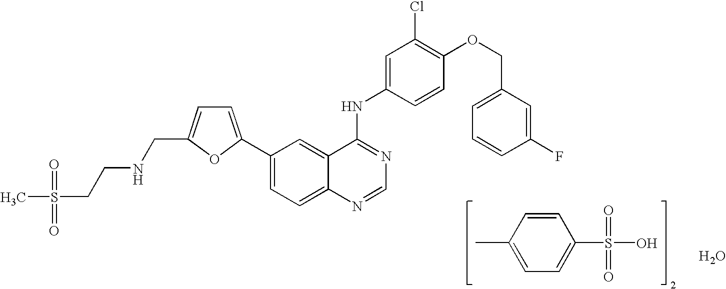

### 1(b) Preparation of monohydrate ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (monohydrate ditosylate salt of compound of formula (II))

### Stage 1: Preparation of N-{3-chloro-4-[(3-fluorobenzyl)oxy]phenyl}-6-iodo-4-quinazolinamine

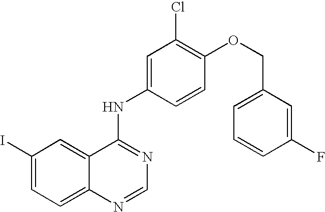

4-Chloro-6-iodoquinazoline (1 wt) was added to a solution of fluorobenzyloxyaniline (0.894 wt, 1.03 equiv) in N-methylpyrrolidinone (8.26 wt, 8 vol) at ca 20° C., and after the initial exotherm had subsided, the resulting solution was stirred at 20°-25° C. for at least 30 minutes. The dark solution was treated with triethylamine (0.58 vol, 1.2 equiv) and the mixture was stirred for 20-30 minutes. Isopropanol (2.5 vol) was added and the mixture was heated to ca 50° C. Water (up to 3 vol) was added slowly to the vessel over 10-15 minutes, while keeping the temperature at ca 50° C. Once crystallisation had commenced the addition was stopped and the resulting slurry was aged for 30-45 minutes at ca 50° C. Any residual water (from the 3 vol) was added, then further water (5 vol) was added to the vessel over ca 30 minutes while maintaining the temperature at ca 50° C. The resulting slurry was cooled to ca 20° C. over ca 30 minutes and aged at ca 20° C. for at least 30 minutes. The solid was collected by filtration and washed sequentially with water (2×5 vol), then isopropanol (5 vol). The product was dried in vacuo at ca 60° C. to give the title compound as a cream crystalline solid.

### Stage 2: Preparation of 5-(4-[3-chloro-4-(3-fluorobenzyloxy)-anilino]-6-quinazolinyl)-furan-2-carbaldehyde 4-methylbenzenesulfonate

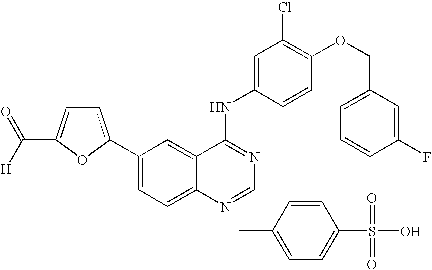

A mixture of N-{3-chloro-4-[(3-fluorobenzyl)oxy]phenyl}-6-iodo-4-quinazolinamine (1 wt), boronic acid (0.37 wt, 1.35 equiv), and 10% palladium on charcoal (0.028 wt, 50% water wet) was slurried in IMS (15 vol). The resultant suspension was stirred for 5 minutes, treated with di-isopropylethylamine (0.39 vol, 1.15 equiv) and then heated to ca 70° C. for ca 3 hours when the reaction was complete (determined by HPLC analysis). The mixture was diluted with tetrahydrofuran (THF, 15 vol) and then filtered (hot—through GFA filter paper) to remove catalyst. The vessel was rinsed with IMS (2 vol).

A solution of p-toluenesulfonic acid monohydrate (1.54 wt, 4.1 equiv) in water (3 vol) was added over 5-10 minutes to the filtered solution maintained at 65° C. After crystallisation the suspension was stirred at 60°-65° C. for 1 hour, cooled to ca 25° C. over 1 hour and stirred at this temperature for a further 2 hours. The solid was collected by filtration, washed with IMS (3 vol) then dried in vacuo at ca 50° C. to give the title compound as a yellow-orange crystalline solid.

### Stage 3: Preparation of anhydrous ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (anhydrous ditosylate salt of compound of formula (II))

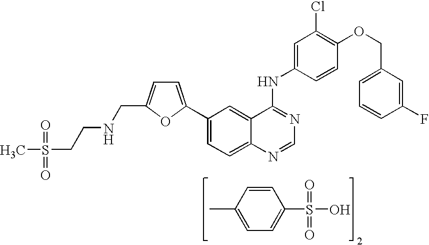

5-(4-[3-chloro-4-(3-fluorobenzyloxy)-anilino]-6-quinazolinyl)-furan-2-carbaldehyde 4-methylbenzenesulfonate (1 wt) and 2-(methylsulfonyl)ethylamine hydrochloride (0.4 wt, 1.6 equiv) were suspended in THF (10 vol). Sequentially, acetic acid (0.35 vol, 4 equiv) and di-isopropylethylamine (1.08 vol, 4 equiv) were added. The resulting solution was stirred at 30°-35° C. for ca 1 hour then cooled to ca 23° C. Sodium triacetoxyborohydride (0.66 wt, 2 equiv) was then added as a continual charge over approximately 15 minutes (some effervescence is seen at this point). The resulting mixture was stirred at ca 22° C. for ca 2 hours then sampled for HPLC analysis. The reaction was quenched by addition of 5M aqueous sodium hydroxide (5 vol) and stirred for ca 30 minutes (some effervescence is seen at the start of the caustic addition).

The aqueous phase was then separated, extracted with THF (2 vol) and the combined THF extracts were then washed with 10% w/v aqueous sodium chloride solution (4 vol). A solution of p-toluenesulfonic acid monohydrate (pTSA, 1.77 wt, 6 equiv) in THF (7 vol)¹ was prepared and warmed to ca 55° C. The THF solution of N-{3-Chloro-4-[(3-fluorobenzyl)oxy]phenyl}-6-[5-({[2-(methanesulphonyl)ethyl]amino}methyl)-2-furyl]-4-quinazolinamine was added to the pTSA solution over at least 30 minutes, maintaining the batch temperature at ca 55°±3° C. 2. The resulting suspension was stirred at ca 55° C. for 2 hours, cooled to 20°-25° C. over ca 60 minutes and aged at this temperature for ca 30 minutes. The solid was collected by filtration, washed with THF (2×2 vol) and dried in vacuo at ca 40° C. to give the desired compound as a pale yellow crystalline solid.

### Stage 4: Preparation of monohydrate ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (monohydrate ditosylate salt of compound of formula (II))

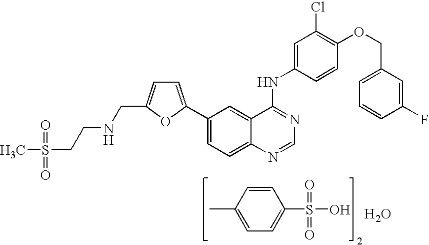

A suspension of the anhydrous ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (1 wt), in tetrahydrofuran (THF, 14 vol) and water (6 vol) was heated to ca 55°-60° C. for 30 minutes to give a solution which was clarified by filtration and the lines washed into the crystallisation vessel with THF/Water (7:3, 2 vol). The resultant solution was heated to reflux and tetrahydrofuran (9 vol, 95% w/w azeotrope with water) was distilled off at atmospheric pressure.

The solution was seeded with N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate (0.002 wt). Once the crystallisation was established water (6 vol) was added while maintaining the reaction temperature above 55° C. The mixture was cooled to 5°-15° C. over ca 2 hours. The solid was collected by filtration, washed with tetrahydrofuran/water (3:7 ratio, 2×2 vol) and dried in vacuo at 45° C. to give N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate as a bright yellow crystalline solid.

### Example 4

### Preparation of (4-(3-Fluoro-benzyloxy-3-bromophenyl)-(6-(5-((2-methanesulphonyl-ethylamino)-methyl)-furan-2-yl)-quinazolin-4-yl)-amine and di-hydrochloride and ditosylate salts thereof

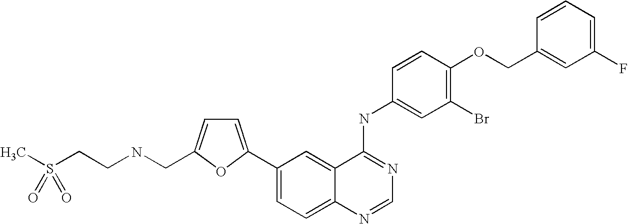

### (a) Preparation of 2-Bromo-4-nitrophenol

2-Bromo-4-nitroanisole (20 g, 0.086 mol) was dissolved in DMF (414 mL) at room temperature under N₂. Sodium ethylthiolate (17.4 g, 0.207 mol) was added and the reaction mixture was warmed to 115° C. for 2 hours. The reaction was cooled to room temperature and diluted with EtOAc (200 mL) and 1 M HCl (aq., 200 mL). The phases were separated, and the desired product was extracted into 1 M NaOH (aq, 150 mL×3). The basic aqueous extracts were combined and acidified using conc. HCl. The desired product was extracted from the acidic aqueous solution using EtOAc (250 mL×2). The combined organic layers were washed with brine and dried over sodium sulfate. The volatiles were removed in vacuo to afford a light brown semi-solid (9.8 g, 52% yield). ¹H NMR (DMSO-d6) δ 8.33 (m, 1H); 8.09 (m, 1H); 7.07 (d, 1H).

### (b) Preparation of 2-Bromo-1-(3-fluorobenzyloxy)-4-nitrobenzene

2-Bromo-4-nitrophenol (4.86 g, 0.0223 mol), triphenylphosphine (7.6 g, 0.0290 mol), 3-fluorobenzylalcohol (3.65 g, 0.0290 mol) were combined and dissolved in THF (89 mL). The reaction temperature was cooled to 0° C. and DIAD (4.50 g, 0.0290 mol) was added. The reaction was allowed to warm slowly to room temperature and stirred for 3 hours before it was diluted with water (100 mL) and EtOAc (100 mL). The layers were separated and the aqueous layer was extracted with EtOAc (200 mL×2). The organic extracts were combined and washed with brine, followed by drying over sodium sulfate. The volatiles were removed in vacuo and the residual semi-solid was treated with diethyl ether. The solids were removed by filtration. The volatiles from the resulting filtrate were removed in vacuo and the material was purified using EtOAc:Hexanes (90/10) in a biotage LC system to afford the title compound as a yellow solid (3.73 g, 68% yield). ¹H NMR (DMSO-d6) δ 8.43 (d, 1H); 8.26 (m, 1H); 7.45 (m, 1H); 7.38 (d, 1H); 7.30 (m, 2H); 7.17 (m, 1H); 5.39 (s, 2H).

### (c) Preparation of 3-Bromo-4-(3-fluorobenzyloxy)-aniline

Under a blanket of N₂, Pt/C (5%, 0.37 g) was charged to a Parr Shaker Flask. Ethanol (150 mL) and 2-bromo-1-(3-fluorobenzyloxy)-4-nitrobenzene (3.73 g, 0.011 mol) were added and the reaction mixture was placed on a Parr Shaker Apparatus under 30 psi of H₂ for 5 h. The reaction was filtered through a pad of Celite to remove the catalyst and the volatiles were removed from the filtrate. The residue was dissolved in the CH₂Cl₂ (5 mL) and treated with conc. HCl (1 mL). The precipitate was collected by filtration and free-based using saturated aqueous sodium bicarbonate (2.27 g, 67% yield) ¹H NMR (DMSO-d6) δ 7.4 (m, 1H); 7.23 (m, 2H); 7.11 (m, 1H); 6.86 (d, 1H); 6.77 (m, 1H); 6.48 (m, 1H); 5.0 (s, 2H); 4.93 (bs, 2H).

### (d) Preparation of 6-Iodo-(4-(3-fluorobenzyloxy)-3-bromophenyl)-quinazolin-4-yl)amine

The title compound was prepared according to Procedure A from 3-bromo-4-(3-fluorobenzyloxy)-aniline (0.79 g, 2.7 mmol) and 4-chloro-6-iodo-quinazoline (0.8 g, 2.7 mmol). ¹H NMR (DMSO-d6) δ 11.1 (bs, 1H); 9.10 (s, 1H); 8.87 (s, 1H); 8.29 (d, 1H); 8.03 (s, 1H); 7.68 (m, 1H); 7.62 (d, 1H); 7.45 (m, 1H); 7.33-7.26 (m, 3H); 7.16 (m, 1H); 5.28 (s, 2H).

### (e) Preparation 5-(4-(3-Bromo-4-(3-fluorobenzyloxy)-anilino)-quinazolin-6-yl)-furan-2-carbaldehyde

The title compound was prepared according to Procedure B followed by Procedure C from 6-iodo-(4-(3-fluorobenzyloxy)-3-bromophenyl)-quinazolin-4-yl)amine (1.0 g, 1.82 mmol) and (1,3 dioxolan-2-yl)-2-(tributylstannyl)furan (1.17 g, 2.73 mmol). ¹H NMR (DMSO-d6) δ 11.89 (bs, 1H); 9.66 (s, 1H); 9.41 (s, 1H); 8.90 (s, 1H); 8.49 (d, 1H); 8.05 (m, 1H); 7.96 (d, 1H); 7.75 (m, 1H); 7.70 (m, 1H); 7.61 (m, 1H); 7.43 (m, 1H); 7.30 (m, 3H); 7.16 (m, 1H); 5.29 (s, 2H).

### (f) Preparation of (4-(3-Fluoro-benzyloxy-3-bromophenyl)-(6-(5-((2-methanesulphonyl-ethylamino)-methyl)-furan-2-yl)-quinazolin-4-yl)-amine dihydrochloride

The title compound was prepared according to Procedure D from a mixture of 5-(4-(3-Bromo-4-(3-fluorobenzyloxy)-anilino)-quinazolin-6-yl)-furan-2-carbaldehyde (0.623 g, 1.2 mmol) in dichloroethane (12 mL), triethylamine (0.167 mL, 1.2 mmol), acetic acid (0.216 mL 3.6 mmol), and 2-methanesulphonylethylamine (0.447 g, 3.6 mmol). The reaction mixture was warmed to reflux for 1 hour and then cooled to rt before adding sodium triacetoxyborohydride (0.5 g). After 0.5 hours of stirring, another aliquot of sodium triacetoxyborohydride (0.5 g) was added and the reaction was stirred an additional 0.5 hours. The reaction was quenched by the addition of a saturated solution of sodium bicarbonate (aq, 50 mL). EtOAc (50 mL) was added and the layers were separated. The organics were washed with brine and dried over sodium sulfate. The volatiles were removed in vacuo. Purification of the compound was achieved using Biotage column chromatography; eluents: CH2Cl2, EtOH, Et3N (150:8:1). The appropriate fractions were combined and the volatiles were removed in vacuo. The compound was crystallized from EtOAc and Et₂O to afford a yellow solid. The hydrochloride salt was made by dissolving the material in a minimal amount of EtOAc and adding 2M HCl in diethyl ether (0.5 mL) to afford a dark yellow solid (0.27 g, 35% yield). ¹H NMR (DMSO-d6) δ 11.70 (bs, 1H); 9.84 (bs, 2H); 9.59 (s, 1H); 8.89 (s, 1H); 8.39 (d, 1H); 8.14 (s, 1H); 7.93 (d, 1H); 7.80 (d, 1H); 7.45 (m, 1H); 7.31 (m, 4H); 7.16 (m, 1H); 6.83 (m, 1H); 5.30 (s, 2H); 4.43 (s, 2H); 3.67 (m, 2H); 3.40 (m, 2H); 3.12 (s, 3H).

### (g) Preparation of (4-(3-Fluoro-benzyloxy)-3-bromophenyl)-(6-(5-((2-methanesulphonyl-ethylamino)-methyl)-furan-2-yl)quinazolin-4-yl)-amine ditosylate

The HCl salt of 5-(4-[3-bromo-4-(3-fluorobenzyloxy)-anilino]-6-quinazolinyl)-furan-2-carbaldehyde, is prepared according to Procedure D and Example 1(e), and is converted to the tosylate salt according to the procedure of Example 1(h). The resultant carbaldehyde tosylate product is used to prepare the (4-(3-Fluoro-benzyloxy)-3-bromophenyl)-(6-(5-((2-methanesulphonyl-ethylamino)-methyl)-furan-2-yl)quinazolin-4-yl)-amine ditosylate according to the procedure of Example 1(i).

### Example 5

### Preparation of (4-(3-Fluoro-benzyloxy)-3-chlorophenyl)-(6-(2-((2-methanesulphonyl-ethylamino)-methyl)-thiazol-4-yl)quinazolin-4-yl)-amine and di-hydrochloride and ditosylate salts thereof

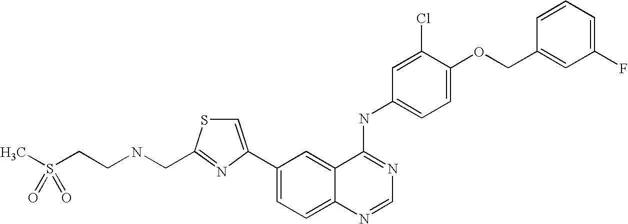

### (a) Preparation of N-(4-(3-fluorobenzyloxy)-chlorophenyl)-6-(1-ethoxyvinylether)-quinazolin-4-yl)-amine

To a suspension of the 6-iodo-(4-(3-fluorobenzyloxy)-3-chlorophenyl)-quinazolin-4-yl amine (12.6 g, 24.93 mmol) in acetonitrile (100 mL) was added tributyl(1-ethoxyvinyl)stannane (9 g, 24.93 mmol) and bis(triphenylphosphine) palladium (II) chloride (1.75 g, 2.29 mmol). The reaction mixture was refluxed for 18 hours, then filtered through a plug of silica gel. The resulting solution was poured into 5% aqueous NH₄OH (200 mL) and extracted with ethyl acetate (500 mL). The organic layer was dried over anhydrous sodium sulfate, concentrated, and purified by silica gel chromatography to provide the title compound as a yellow solid (7.2 g, 64% yield). ¹H NMR (400 MHz, d₆ DMSO) δ 9.92 (s, 1H), 8.76 (s, 1H), 8.58 (s, 1H), 8.08 (m, 1H), 8.01 (m, 1H), 7.76 (m, 2H), 7.48 (m, 1H), 7.32 (m, 3H), 7.22 (m, 1H), 5.28 (s, 2H), 5.02 (s, 1H), 4.56 (s, 1H), 4.01 (q, 2H), 1.42 (t, 3H); ESI-MS m/z 449.9 (M+H)⁺.

### (b) Preparation of N-{4-[(3-fluorobenzyloxy)]-chlorophenyl}-6-[2-({[2-(methanesulphonyl)ethyl]-[trifluoroacetyl]amino}methyl)-1,3-thiazol-4-yl]-quinazolin-4-yl)-amine

To a solution of N-(4-(3-fluorobenzyloxy)-chlorophenyl)-6-(1-ethoxyvinylether)-quinazolin-4-yl)-amine (7.1 g, 15.8 mmol) in a THF (150 mL)/H₂O (5 mL) mixture cooled to 0° C. was added N-bromosuccinimide (2.81 g, 15.8 mmol). The resulting mixture was stirred for 0.25 hours, then dried over anhydrous sodium sulfate and concentrated. The crude N-(4-(3-fluorobenzyloxy)-chlorophenyl)-6-(bromomethylketone)-quinazolin-4-yl)-amine and N-(trifluoroacetyl)-N-(methanesulphonylethyl)-aminomethylthioamide (4.61 g, 15.8 mmol) were dissolved in DMF (50 mL) and heated at 70° C. for 1 hour. The reaction mixture was concentrated, then diluted with dichloromethane (300 mL) and washed with saturated sodium bicarbonate solution (100 mL). The organic layer was dried over anhydrous sodium sulfate, concentrated, and purified by silica gel chromatography to provide the title compound as a foam (4.6 g, 42% yield). ESI-MS m/z 694.1 (M+H)⁺.

### (c) Preparation of N-{4-[(3-fluorobenzyloxy)]-chlorophenyl}-6-[2-({[2-(methanesulphonyl)ethyl]-amino}methyl)-1,3-thiazol-4-yl]-quinazolin-4-yl)-amine hydrochloride

To a solution of N-{4-[(3-fluorobenzyloxy)]-chlorophenyl}-6-[2-({[2-(methanesulphonyl)ethyl]-[trifluoroacetyl]amino}methyl)-1,3-thiazol-4-yl]-quinazolin-4-yl)-amine (4.6 g, 6.63 mmol) in methanol (100 mL) was added 2M NaOH (50 mL). The resulting mixture was stirred at room temperature for 2 hours, concentrated to ½ volume, poured into H₂O (100 mL), and extracted with dichloromethane (300 mL). The organic layer was dried over anhydrous sodium sulfate, concentrated, and purified by silica gel chromatography. The resulting amine was dissolved in dichloromethane/methanol (3:1, 100 mL) and then 4M HCl/dioxane (20 mL) was added. The resulting mixture was concentrated and filtered to provide the title compound as a yellow solid (4.0 g, 90% yield). ¹H NMR (400 MHz, d₄ MeOH) δ 9.38 (s, 1H), 8.82 (s, 1H), 8.78 (d, 1H), 8.36 (s, 1H), 7.94 (s, 1H), 7.92 (d, 1H), 7.63 (m, 1H), 7.41 (m, 1H), 7.26 (m, 1H), 7.22 (m, 2H), 7.04 (m, 1H), 5.24 (s, 2H), 4.82 (s, 2H), 3.84 (m, 2H), 3.76 (m, 2H), 3.12 (s, 3H); ESI-MS m/z 597.1 (M+H)⁺.

### (d) Preparation of (4-(3-Fluoro-benzyloxy)-3-chlorophenyl)-(6-(2-((2-methanesulphonyl-ethylamino)-methyl)-thiazol-4-yl)quinazolin-4-yl)-amine ditosylate

The HCL salt of (4-(3-Fluoro-benzyloxy)-3-chlorophenyl)-(6-(2-((2-methanesulphonyl-ethylamino)-methyl)-thiazol-4-yl)quinazolin-4-yl)-amine was prepared according to Procedures 3(a) to (c) and then converted to the (4-(3-Fluoro-benzyloxy)-3-chlorophenyl)-(6-(2-((2-methanesulphonyl-ethylamino)-methyl)-thiazol-4-yl)quinazolin-4-yl)-amine ditosylate salt according to the procedure of Examples 1 and 2. ¹H NMR (300 MHz, d6-DMSO) 11.4 (brs, 1H), 9.51 (brs, 1H), 9.24 (s, 1H), 8.95 (s, 1H), 8.68 (d, J=9 Hz, 1H), 8.42 (s, 1H), 7.96 (d, J=9 Hz, 1H), 7.89 (d, J=2 Hz, 1H), 7.64 (dd, J=2, 9 Hz, 1H), 7.47 (m, 5H), 7.34 (m, 3H), 7.20 (t, J=9 Hz, 1H), 7.10 (d, J=8 Hz, 4H), 5.32 (s, 2H), 4.76 (d, 2H), 3.61 (s, 4H), 3.15 (s, 3H), 2.28 (s, 6H).

### Biological Data

### Tumor Studies: BT474

The BT474 xenografts were maintained by serial transplantation of tumor fragments in SCID mice. Tumors are initiated by injection of tumor fragments subcutaneously in the axillary region.

### Tumor Studies: NCI H322

NCI H322 cells obtained from the ATCC and were cultured in RPMI 1640+10% Fetal bovine serum, Sodium pyruvate and L-Glutamine at 370 in a 95/5% air/CO₂ atmosphere. Cells were harvested following trypsin digestion and brought to a density of 2×10⁶ cells/200 μl in PBS. Tumors were initiated by injection of the cell suspension subcutaneously in the axillary region. In addition some experiments were performed following serial transplantation of tumor fragments in SCID mice. Tumors were initiated by injection of tumor fragments subcutaneously in the axillary region.

### Tumor Studies: Measurements

For the xenograft models used here, solid tumors were measured by electronic caliper measurement through the skin. Measurements were typically made twice weekly.

### Tumor Studies Formulation and Administration

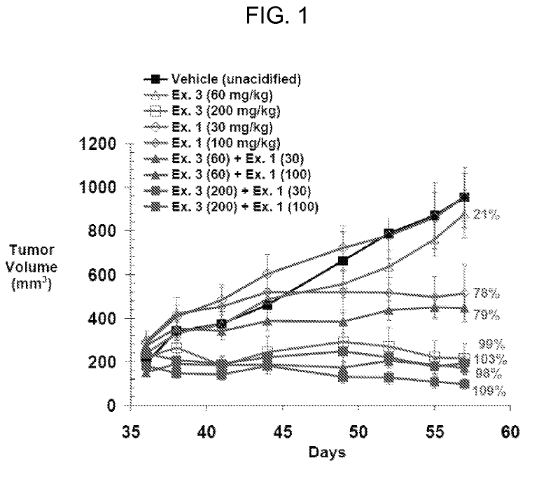

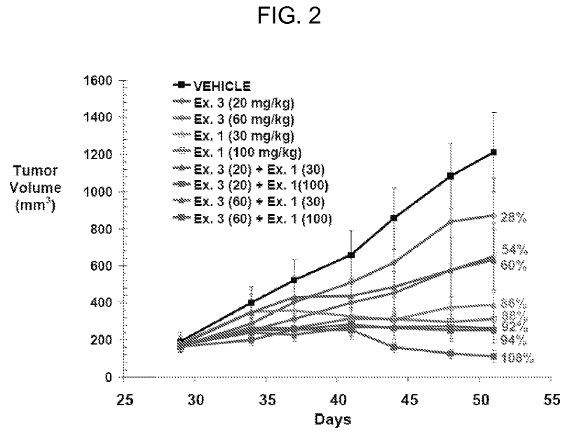

Drugs were administered by P.O. route. The compounds or salts thereof of Example 1 and Example 3 were formulated in aqueous 0.5% hydroxypropyl methylcellulose, 0.1% Tween 80 and administered as a suspension once daily for 21 days as indicated in the respective tables and figures. These studies were performed under IACUC #468 and 603. The results are illustrated in Tables 1-4, and FIGS. 1 and 2. FIGS. 1 and 2 are the graphical representation of the data contained in Tables 3 and 4, respectively.

Table 1 summarizes the results of two independent experiments of dosing of a BT474 (breast) subcutaneous (s.c.) human xenograft mouse model with the compound of Example 1 and/or the compound of Example 3. The column labeled “Exp. 1” contains the results presented in FIG. 1 for the last data point on the graph. The column labeled “Exp. 2” contains analogous results from an independent experiment. Data are presented as percent inhibition of tumor growth, as compared to vehicle.

TABLE 1

| | Exp. 1 | Exp. 2 |
|---|---|---|
| Example 3 (60 mg/kg) | 21 | 58* |
| Example 3 (200 mg/kg) | 99* | 85* |
| Example 1 (30 mg/kg) | 16 | 47* |
| Example 1 (100 mg/kg) | 78* | 64* |
| Ex. 1 (30 mg/kg) + Ex. 3 (60 mg/kg) | 79* | 71* |
| Ex. 1 (100 mg/kg) + Ex. 3 (60 mg/kg) | 98* | 84* |
| Ex. 1 (30 mg/kg) + Ex. 3 (200 mg/kg) | 103* | 91* |
| Ex. 1 (100 mg/kg) + Ex. 3 (200 mg/kg) | 109* | 88* |

*Significant difference from vehicle treated group (paired comparison of slopes using Tukey-Kramer HSD)

Table 2 summarizes the results of three independent experiments of dosing of a NCI H322 (non-small cell lung carcinoma) subcutaneous human xenograft mouse model with the compound of Example 1 and/or the compound of Example 3. The column labeled “Exp. 3” contains the results presented in FIG. 2 for the last data point on the graph. The columns labeled “Exp. 1” and “Exp. 2” contain analogous results from independent experiments. Data are presented as percent inhibition of tumor growth, as compared to vehicle. N.D. indicates that experiment was not done.

TABLE 2

| | Exp. 1 | Exp. 2 | Exp. 3 |
|---|---|---|---|
| Example 3 (20 mg/kg) | N.D | 31 | 28 |
| Example 3 (60 mg/kg) | 75* | N.D. | 54 |
| Example 1 (30 mg/kg) | 34 | 20 | 86* |
| Example 1 (100 mg/kg) | 56* | 62 | 88* |
| Ex. 1 (30 mg/kg) + Ex. 3 (20 mg/kg) | N.D. | 43 | 60* |
| Ex. 1 (100 mg/kg) + Ex. 3 (20 mg/kg) | N.D. | 69* | 94* |
| Ex. 1 (30 mg/kg) + Ex. 3 (60 mg/kg) | 81* | N.D. | 92* |
| Ex. 1 (100 mg/kg) + Ex. 3 (60 mg/kg) | 81* | N.D. | 108* |

*Significant difference from vehicle treated group (paired comparison of slopes using Tukey-Kramer HSD)

Table 3 contains the mean tumor volume and standard error of the mean (S.E.M.) for each data point for the graph in FIG. 1. Mean tumor volume is shown in mm³.

Table 4 contains the mean tumor volume and standard error of the mean (S.E.M.) for each data point for the graph in FIG. 2. Mean tumor volume is shown in mm³.

TABLE 3

| | Day 36 | | Day 38 | | Day 41 | | Day 44 | | Day 49 | | Day 52 | | Day 55 | | Day 57 | |
|---|---|---|---|---|---|---|---|---|---|---|---|---|---|---|---|---|
| | Mean | S.E.M. | Mean | S.E.M. | Mean | S.E.M. | Mean | S.E.M. | Mean | S.E.M. | Mean | S.E.M. | Mean | S.E.M. | Mean | S.E.M. |
| Vehicle | 193 | 27.0 | 340 | 45.7 | 373 | 59.2 | 460 | 81.6 | 663 | 140.4 | 788 | 170.8 | 869 | 162.1 | 953 | 147.1 |
| (unacidified) | | | | | | | | | | | | | | | | |
| Ex. 3, | 235 | 16.6 | 328 | 31.9 | 367 | 30.9 | 485 | 49.1 | 554 | 63.9 | 633 | 84.2 | 763 | 83.3 | 878 | 116.5 |
| 60 mg/kg | | | | | | | | | | | | | | | | |
| Ex. 3, | 219 | 40.8 | 262 | 68.7 | 184 | 45.0 | 242 | 80.0 | 288 | 97.0 | 270 | 90.3 | 219 | 82.3 | 214 | 75.3 |
| 200 mg/kg | | | | | | | | | | | | | | | | |
| Ex. 1, | 281 | 37.9 | 406 | 56.8 | 481 | 74.4 | 601 | 95.5 | 722 | 106.8 | 777 | 112.3 | 860 | 124.1 | 954 | 116.3 |
| 30 mg/kg | | | | | | | | | | | | | | | | |
| Ex. 1, | 293 | 49.7 | 417 | 84.4 | 453 | 90.5 | 519 | 101.3 | 519 | 103.9 | 515 | 74.2 | 496 | 101.1 | 513 | 139.0 |
| 100 mg/kg | | | | | | | | | | | | | | | | |
| Ex. 3 (60) + | 272 | 24.3 | 353 | 44.8 | 338 | 43.0 | 386 | 51.1 | 383 | 57.0 | 434 | 48.4 | 450 | 58.3 | 445 | 61.7 |
| Ex. 1 (30) | | | | | | | | | | | | | | | | |
| Ex. 3 (60) + | 149 | 20.0 | 190 | 25.3 | 182 | 23.3 | 186 | 31.3 | 173 | 27.6 | 202 | 34.3 | 186 | 29.7 | 168 | 30.3 |
| Ex. 1 (100) | | | | | | | | | | | | | | | | |
| Ex. 3 (200) + | 236 | 31.9 | 206 | 36.3 | 188 | 32.1 | 221 | 38.4 | 245 | 47.2 | 218 | 42.8 | 177 | 35.5 | 191 | 34.5 |
| Ex. 1 (30) | | | | | | | | | | | | | | | | |
| Ex. 3 (200) + | 179 | 32.8 | 147 | 24.8 | 141 | 31.3 | 179 | 39.6 | 131 | 34.3 | 125 | 31.5 | 105 | 28.8 | 96 | 25.6 |
| Ex. 1 (100) | | | | | | | | | | | | | | | | |

TABLE 4

| | Day 29 | | Day 34 | | Day 37 | | Day 41 | | Day 44 | | Day 48 | | Day 51 | |
|---|---|---|---|---|---|---|---|---|---|---|---|---|---|---|
| | Mean | S.E.M. | Mean | S.E.M. | Mean | S.E.M. | Mean | S.E.M. | Mean | S.E.M. | Mean | S.E.M. | Mean | S.E.M. |
| Vehicle | 190 | 53.3 | 399 | 88.4 | 522 | 110.8 | 659 | 130.0 | 855 | 165.1 | 1084 | 176.4 | 1213 | 212.9 |
| (unacidified) | | | | | | | | | | | | | | |
| Ex. 3, 20 mg/kg | 167 | 29.7 | 288 | 45.5 | 403 | 73.5 | 511 | 148.3 | 616 | 184.3 | 837 | 225.2 | 871 | 199.3 |
| Ex. 3, 60 mg/kg | 175 | 40.2 | 252 | 66.8 | 313 | 85.7 | 400 | 100.2 | 451 | 116.3 | 577 | 177.6 | 649 | 182.3 |
| Ex. 1, 30 mg/kg | 182 | 41.5 | 351 | 121.9 | 358 | 101.9 | 329 | 39.7 | 309 | 34.5 | 377 | 60.8 | 389 | 66.8 |
| Ex. 1, 100 mg/kg | 166 | 28.6 | 268 | 54.9 | 266 | 59.9 | 316 | 61.5 | 312 | 57.8 | 297 | 64.6 | 313 | 67.2 |
| Ex. 3 (20) + | 172 | 34.7 | 350 | 91.4 | 433 | 114.7 | 437 | 96.9 | 486 | 133.0 | 576 | 143.9 | 633 | 163.7 |
| Ex. 1 (30) | | | | | | | | | | | | | | |
| Ex. 3 (20) + | 174 | 30.7 | 251 | 40.5 | 258 | 45.8 | 281 | 40.7 | 264 | 39.2 | 254 | 59.1 | 254 | 69.7 |
| Ex. 1 (100) | | | | | | | | | | | | | | |
| Ex. 3 (60) + | 172 | 37.4 | 241 | 46.5 | 227 | 32.7 | 269 | 64.5 | 269 | 67.1 | 273 | 65.0 | 263 | 61.5 |
| Ex. 1 (30) | | | | | | | | | | | | | | |
| Ex. 3 (60) + | 165 | 31.2 | 202 | 30.8 | 259 | 45.6 | 255 | 33.7 | 161 | 27.1 | 127 | 26.1 | 112 | 32.5 |
| Ex. 1 (100) | | | | | | | | | | | | | | |

FIG. 1 is a representative experiment and illustrates dosing of a BT474 (breast) subcutaneous human xenograft mouse model with the compound of Example 1 and/or the compound of Example 3. The compound of Example 1 as a monotherapy in the BT474 s.c. human xenograft mouse model showed some anti-tumor activity (about 16-78% tumor growth inhibition). Dosing of the compound of Example 3 also showed anti-tumor activity in the same model as monotherapy (about 21-99% tumor growth inhibition at highest dose). When the compound of Example 1 and the compound of Example 3 were used in combination, 79-109% tumor growth inhibition was observed during treatment.

FIG. 2 illustrates dosing of a NCI H322 (non-small cell lung carcinoma) subcutaneous human xenograft mouse model with the compound of Example 1 and/or the compound of Example 3. The compound of Example 1 dosed as monotherapy in the NCI H322 s.c. human xenograft mouse model showed some anti-tumor activity (about 86-88% tumor growth inhibition). The compound of Example 3 also showed anti-tumor activity in the same model when dosed as monotherapy (about 28-54% tumor growth inhibition at the highest dose tested). When the compound of Example 1 and the compound of Example 3 were used in combination, 60-108% anti-tumor activity was observed during treatment.

What is claimed is:

1. A method of treating cancer in a mammal, comprising: administering to said mammal
(a) a compound of formula I 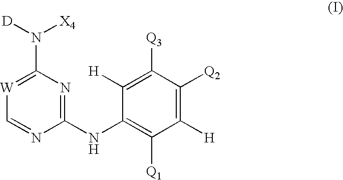
or a salt, solvate, or physiologically functional derivative thereof;
wherein:
D is 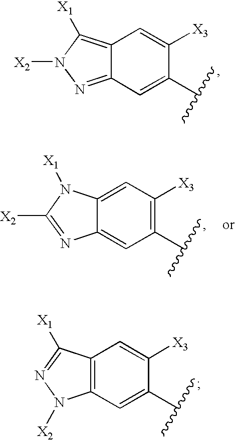
X₁ is hydrogen, C₁₋₄ alkyl, C₁₋₄ haloalkyl, or C₁₋₄ hydroxyalkyl;
X₂ is hydrogen, C₁₋₄ alkyl, C₁₋₄ haloalkyl, C(O)R¹, or aralkyl;
X₃ is hydrogen or halogen;
X₄ is hydrogen, C₁₋₄ alkyl, C₁₋₄ haloalkyl, heteroaralkyl, cyanoalkyl, —(CH₂)ₚC═CH(CH₂)ₜH, —(CH₂)ₚC≡C(CH₂)ₜH, or C₃₋₇ cycloalkyl;
p is 1, 2, or 3;
t is 0 or 1;
W is N or C—R, wherein R is hydrogen, halogen, or cyano;
Q₁ is hydrogen, halogen, C₁₋₂ haloalkyl, C₁₋₂ alkyl, C₁₋₂ alkoxy, or C₁₋₂ haloalkoxy;
Q₂ is A¹ or A²;
Q₃ is A¹ when Q₂ is A² and Q₃ is A² when Q₂ is A¹;
wherein
A¹ is hydrogen, halogen, C₁₋₃ alkyl, C₁₋₃ haloalkyl, —OR¹, and
A² is the group defined by -(Z)ₘ-(Z¹)-(Z²), wherein
Z is CH₂ and m is 0, 1, 2, or 3, or
Z is NR² and m is 0 or 1, or
Z is oxygen and m is 0 or 1, or
Z is CH₂NR² and m is 0 or 1;
Z¹ is S(O)₂, S(O), or C(O); and
Z² is C₁-C₄ alkyl, NR³R⁴, aryl, arylamino, aralkyl, aralkoxy, or heteroaryl;
R¹ is C₁₋₄ alkyl;
R², R³, and R⁴ are each independently selected from hydrogen, C₁₋₄ alkyl, C₃₋₇ cycloalkyl, —S(O)₂R⁵, and —C(O)R⁵;
R⁵ is C₁₋₄ alkyl, or C₃₋₇ cycloalkyl; and
when Z is oxygen then Z¹ is S(O)₂ and when D is 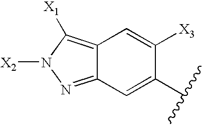
then X₂ is C₁₋₄ alkyl, C₁₋₄ haloalkyl, C(O)R¹, or aralkyl; and
(b) a compound of formula II 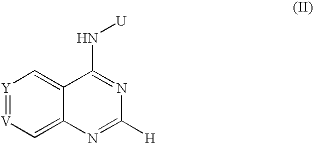
or a salt, solvate, or physiologically functional derivative thereof;
wherein
Y is CR⁶ and V is N;
or Y is CR⁶ and V is CR⁷;
R⁶ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R⁷ is selected from the group consisting of hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R⁸ group and optionally substituted by at least one independently selected R⁹ group;
R⁸ is selected from the group consisting of benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R⁸ represents trihalomethylbenzyl or trihalomethylbenzyloxy;
or R⁸ represents a group of formula 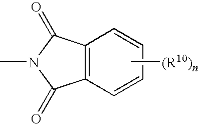
wherein each R¹⁰ is independently selected from halogen, C₁₋₄ alkyl and C₁₋₄ alkoxy;
and n is 0 to 3; and
each R⁹ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl.

2. The method of claim 1, wherein (a) the compound of formula I is a compound of formula I^{a} 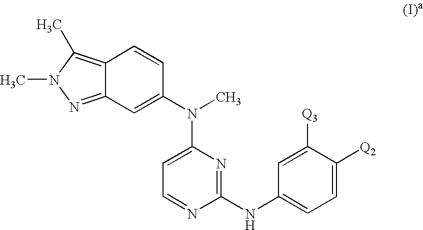
or a salt, solvate or physiologically functional derivative thereof;
wherein Q₃ is A¹ when Q₂ is A² and Q₃ is A² when Q₂ is A¹;
wherein
A¹ is hydrogen, halogen, C₁₋₃ alkyl, and
A² is the group defined by -(Z)ₘ-(Z¹)-(Z²), wherein
Z is CH₂ and m is 0, 1, 2, or 3;
Z¹ is S(O)₂, S(O), or C(O); and
Z² is C₁₋₄ alkyl, or NR³R⁴;
R³ and R⁴ are each independently selected from hydrogen, or C₁₋₄ alkyl; and
(b) the compound of formula II is a compound of formula II^{a} 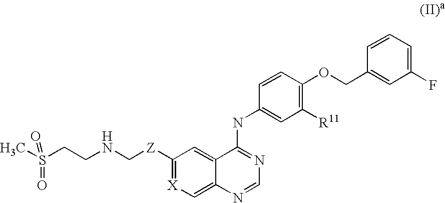
or a salt, solvate or physiologically functional derivative thereof; wherein R¹¹ is —Cl or —Br, X is CH, N, or CF, and Z is thiazole or furan.

3. The method of claim 1, wherein (a) the compound of formula I is a compound of formula I^{b} 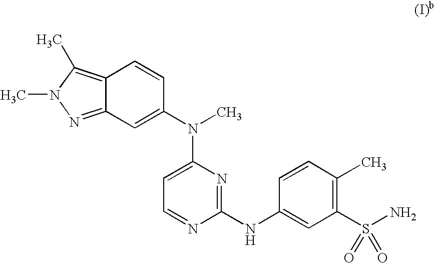
or a salt, solvate, or physiological functional derivative thereof; and
(b) the compound of formula II is a compound of formula II^{b} 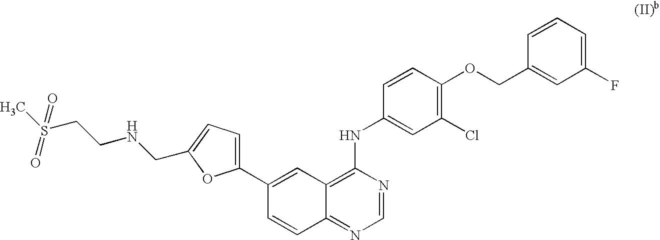
or a salt, solvate, or physiological functional derivative thereof.

4. The method of claim 1, wherein (a) the compound of formula I is a monohydrochloride salt of a compound of formula I^{b} 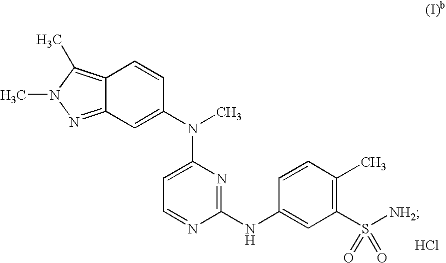
and
(b) the compound of formula II is a monohydrate ditosylate salt of a compound of formula II^{b}

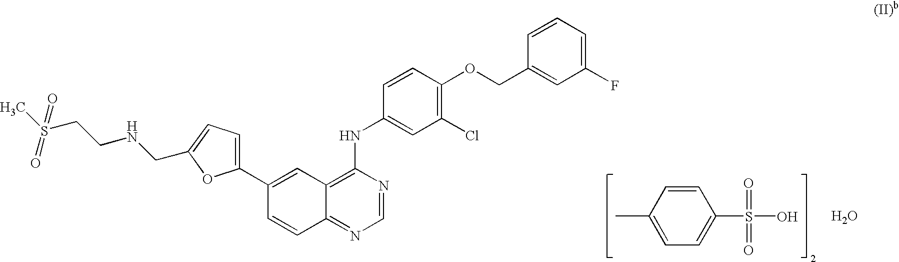

5. The method of claim 1, wherein the compound of formula I is a monohydrochloride salt of a compound of formula I^{b} 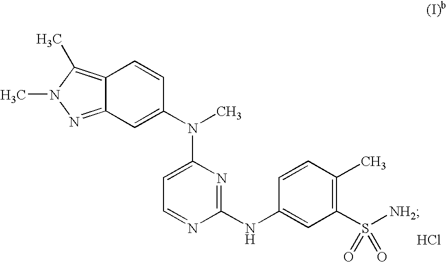
and
(b) the compound of formula II is an anhydrous ditosylate salt of a compound of formula II^{b}

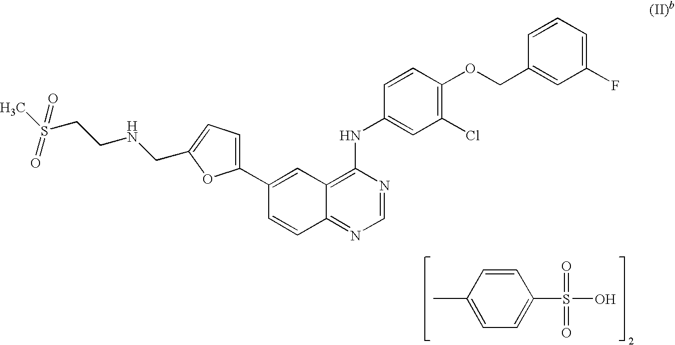

6. A pharmaceutical composition comprising:
(a) a compound of formula I 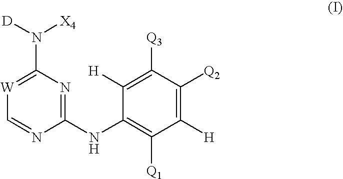
or a salt, solvate, or physiologically functional derivative thereof;
wherein:
D is 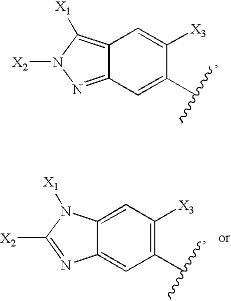 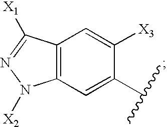
X₁ is hydrogen, C₁₋₄ alkyl, C₁₋₄ haloalkyl, or C₁₋₄ hydroxyalkyl;
X₂ is hydrogen, C₁₋₄ alkyl, C₁₋₄ haloalkyl, C(O)R¹, or aralkyl;
X₃ is hydrogen or halogen;
X₄ is hydrogen, C₁₋₄ alkyl, C₁₋₄ haloalkyl, heteroaralkyl, cyanoalkyl, —(CH₂)ₚC═CH(CH₂)ₜH, —(CH₂)ₚC≡C(CH₂)ₜH, or C₃₋₇ cycloalkyl;
p is 1, 2, or 3;
t is 0 or 1;
W is N or C—R, wherein R is hydrogen, halogen, or cyano;
Q₁ is hydrogen, halogen, C₁₋₂ haloalkyl, C₁₋₂ alkyl, C₁₋₂ alkoxy, or C₁₋₂ haloalkoxy;
Q₂ is A¹ or A²;
Q₃ is A¹ when Q₂ is A² and Q₃ is A² when Q₂ is A¹;
wherein
A¹ is hydrogen, halogen, C₁₋₃ alkyl, C₁₋₃ haloalkyl, —OR¹, and
A² is the group defined by -(Z)ₘ-(Z¹)-(Z²), wherein
Z is CH₂ and m is 0, 1, 2, or 3, or
Z is NR² and m is 0 or 1, or
Z is oxygen and m is 0 or 1, or
Z is CH₂NR² and m is 0 or 1;
Z¹ is S(O)₂, S(O), or C(O); and
Z² is C₁₋₄ alkyl, NR³R⁴, aryl, arylamino, aralkyl, aralkoxy, or heteroaryl;
R¹ is C₁₋₄ alkyl;
R², R³, and R⁴ are each independently selected from hydrogen, C₁₋₄ alkyl, C₃₋₇ cycloalkyl, —S(O)₂R⁵, and —C(O)R⁵;
R⁵ is C₁₋₄ alkyl, or C₃₋₇ cycloalkyl; and
when Z is oxygen then Z¹ is S(O)₂ and when D is 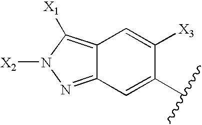
then X₂ is C₁₋₄ alkyl, C₁₋₄ haloalkyl, C(O)R¹, or aralkyl; and
(b) a compound of formula II 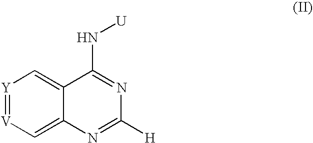
or a salt, solvate, or physiologically functional derivative thereof;
wherein
Y is CR⁶ and V is N;
or Y is CR⁶ and V is CR⁷;
R⁶ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R⁷ is selected from the group consisting of hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R⁸ group and optionally substituted by at least one independently selected R⁹ group;
R⁸ is selected from the group consisting of benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R⁸ represents trihalomethylbenzyl or trihalomethylbenzyloxy;
or R⁸ represents a group of formula 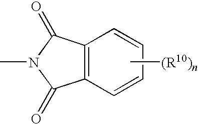
wherein each R¹⁰ is independently selected from halogen, C₁₋₄ alkyl and C₁₋₄ alkoxy;
and n is 0 to 3; and
each R⁹ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl.

7. The pharmaceutical composition of claim 6, wherein (a) the compound of formula I is a compound of formula I^{a} 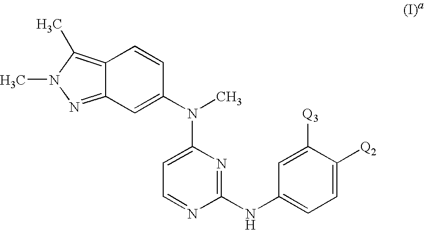
or a salt, solvate or physiologically functional derivative thereof;
wherein Q₃ is A¹ when Q₂ is A² and Q₃ is A² when Q₂ is A¹;
wherein
A¹ is hydrogen, halogen, C₁₋₃ alkyl, and
A² is the group defined by -(Z)ₘ-(Z¹)-(Z²), wherein
Z is CH₂ and m is 0, 1, 2, or 3;
Z¹ is S(O)₂, S(O), or C(O); and
Z² is C₁₋₄ alkyl, or NR³R⁴;
R³ and R⁴ are each independently selected from hydrogen, or C₁₋₄ alkyl; and
(b) the compound of formula II is a compound of formula II^{a} 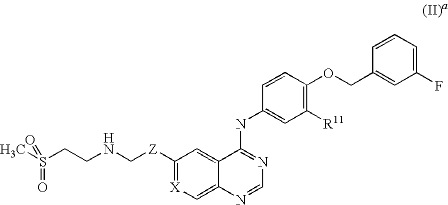
or a salt, solvate or physiologically functional derivative thereof; wherein R¹¹ is —Cl or —Br, X is CH, N, or CF, and Z is thiazole or furan.

8. The pharmaceutical composition of claim 6, wherein (a) the compound of formula I is a compound of formula I^{b} 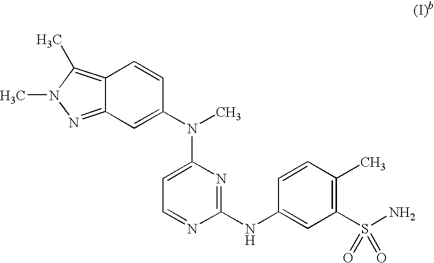
or a salt, solvate, or physiological functional derivative thereof; and
(b) the compound of formula II is a compound of formula II^{b} 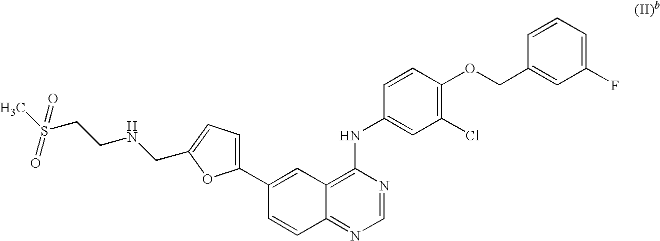
or a salt, solvate, or physiological functional derivative thereof.

9. The pharmaceutical composition of claim 6, wherein (a) the compound of formula I is a monohydrochloride salt of a compound of formula I^{b} 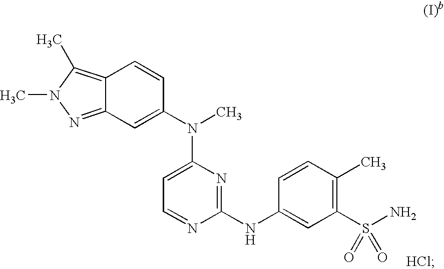
and
(b) the compound of formula II is a monohydrate ditosylate salt of the compound of formula II^{b}

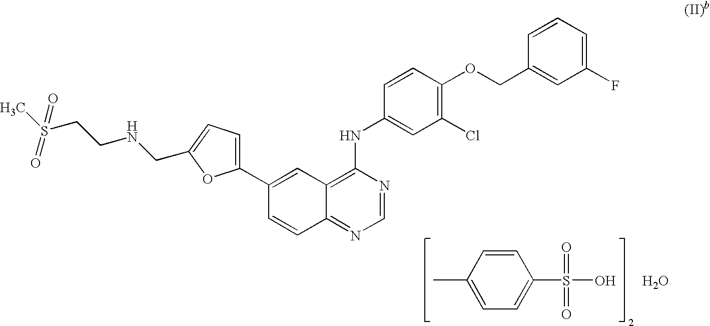

10. The pharmaceutical composition of claim 6, wherein (a) the compound of formula I is a monohydrochloride salt of a compound of formula I^{b} 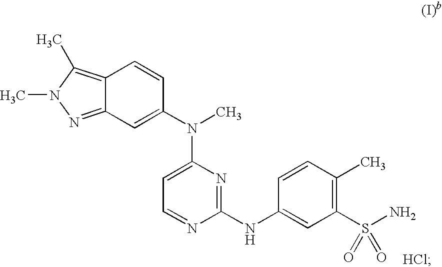
and
(b) the compound of formula II is an anhydrous ditosylate salt of the compound of formula II^{b}

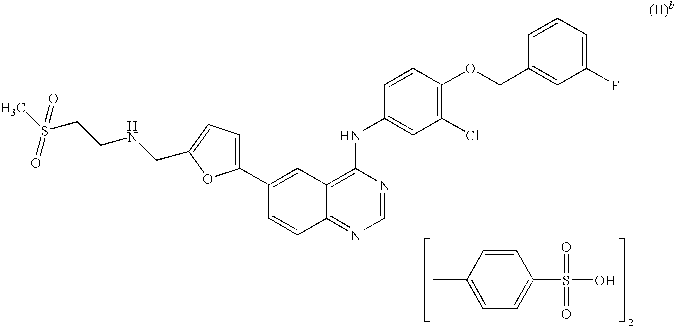

11. A pharmaceutical combination comprising: a compound of formula I, I^{a} or I^{b} or salt, solvate or physiologically functional derivative thereof, and a compound of formula II, II^{a} or II^{b} or salt, solvate or physiologically functional derivative thereof for use in therapy.

12. The use of a pharmaceutical combination comprising: a compound of formula I, I^{a} or I^{b} or salt, solvate or physiologically functional derivative thereof, and a compound of formula II, II^{a} or II^{b} or salt, solvate or physiologically functional derivative thereof for the preparation of a medicament useful in the treatment of cancer.
